SEQUENCE LISTING (13 sequences)

SEQ ID NO: 1 - protein, 16 aa, synthetic construct
  source 1..16: note = 1..16
  REGION 1..16: note = Linear Epitope
RRTEEENLRKKGEPHH

SEQ ID NO: 2 - protein, 16 aa, synthetic construct
  REGION 1..16: note = Immunization Peptide
CRTEEENLRKKGEPHH

SEQ ID NO: 3 - protein, 392 aa, synthetic construct
  REGION 1..392: note = p53 Protein
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 4 - protein, 572 aa, synthetic construct
  REGION 1..572: note = Heavy chain of 2D3A8 antibody
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 5 - protein, 214 aa, synthetic construct
  REGION 1..214: note = Light chain of 2D3A8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - protein, 117 aa, synthetic construct
  REGION 1..117: note = Heavy chain variable region of 2D3A8 antibody
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSS

SEQ ID NO: 7 - protein, 107 aa, synthetic construct
  REGION 1..107: note = Light chain variable region of 2D3A8 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 8 - protein, 5 aa, synthetic construct
  REGION 1..5: note = Heavy chain CDR1 of 2D3A8 antibody
SYVMH

SEQ ID NO: 9 - protein, 17 aa, synthetic construct
  REGION 1..17: note = Heavy chain CDR2 of 2D3A8 antibody
YINPYNDGTKYNEKFKG

SEQ ID NO: 10 - protein, 8 aa, synthetic construct
  REGION 1..8: note = Heavy chain CDR3 of 2D3A8 antibody
GGYYAMDY

SEQ ID NO: 11 - protein, 11 aa, synthetic construct
  REGION 1..11: note = Light chain CDR1 of 2D3A8 antibody
RASQDISNYLN

SEQ ID NO: 12 - protein, 7 aa, synthetic construct
  REGION 1..7: note = Light chain CDR2 of 2D3A8 antibody
YTSRLHS

SEQ ID NO: 13 - protein, 9 aa, synthetic construct
  REGION 1..9: note = Light chain CDR3 of 2D3A8 antibody
QQGNTLPYT

(19) United States
(12) Patent Application Publication
(10) Pub. No.: US 2025/0172555 A1
(43) Pub. Date: May 29, 2025

(54) DEVICE FOR DETECTION AND PROGNOSTIC ASSESSMENT OF NEURODEGENERATIVE DISORDERS

(71) Applicant: Diadem SpA, Milan (IT)

(72) Inventors: Paul Kinnon, Milan (IT); Simona Piccirella, Milan (IT); James Clark, Cambridge (GB)

(21) Appl. No.: 19/040,195

(22) Filed: Jan. 29, 2025

Related U.S. Application Data
(63) Continuation of application No. PCT/US23/71555, filed on Aug. 2, 2023.
(60) Provisional application No. 63/370,151, filed on Aug. 2, 2022.

(51) Int. Cl. G01N 33/543; G01N 33/68

(52) U.S. Cl. CPC G01N 33/54388; G01N 33/6896; G01N 2333/4748; G01N 2800/2814; G01N 2800/50

(57) ABSTRACT

The present invention relates to a lateral flow test device capable of detecting the presence or absence of unfolded p53 in a liquid sample, such as a blood sample. Also provided are methods of using such a device for quantitative or qualitative measurement of U-p53 in a liquid sample. Detection of the presence of this analyte in the sample identifies if the subject has a risk to develop Alzheimer's Disease, and also is useful to confirm a diagnosis of Alzheimer's disease.

### RELATED APPLICATIONS

This application is a continuation application of International Patent Application No. PCT/US2023/071555, filed Aug. 2, 2023, which claims priority to U.S. Application No. 63/370,151 filed on Aug. 2, 2022, the content of each of which is herein incorporated by reference in its entirety.

### FIELD OF THE INVENTION

The present invention is directed to devices and methods for the detection of an analyte such as a protein in a biological sample such as blood.

### SEQUENCE LISTING

This application contains a Sequence Listing which has been submitted electronically as a WIPO Standard ST.26 XML file via Patent Center, created on Jan. 29, 2025, is entitled “121689-10404.xml” and is 18,948 bytes in size. The sequence listing is incorporated herein by reference in its entirety.

### BACKGROUND

Conventional lateral devices are made of nitrocellulose strip or nylon which are immobilized with binding agents that bind the analyte being tested. Either competitive or non-competitive assays can be performed with the conventional lateral flow test devices for detecting the presence of an analyte in a liquid sample.

The use of reagent-impregnated test strips in specific binding assays, such as immunoassays is known. In such procedures a sample is applied to one portion of the test strip and is allowed to permeate through the strip material, usually with the aid of an eluting solvent such as water or buffer. In so doing, the sample progresses into or through a detection zone in the test strip wherein a specific binding reagent is immobilized. The analyte present in the sample can participate in a sandwich or a competition reaction within the detection zone, with a labelled reagent which can also be incorporated in the test strip or applied thereto.

Some examples of lateral flow devices are disclosed in U.S. Pat. No. 5,075,078, U.S. Pat. No. 5,120,643, U.S. Pat. No. 7,407,813 and U.S. Pat. No. 8,071,394. These prior art devices and methods are not particularly useful for measuring analytes such as isoform of p53 protein at low concentrations with high degree of accuracy to enable diagnosis and risk factor analysis for neurological diseases.

### SUMMARY OF DISCLOSURE

In one aspect, the disclosure provides a device for detecting the presence of unfolded p53 protein in a biological sample, the device comprising a test strip defining a flow path comprising a sample zone, a reagent zone, and detection zone: wherein said sample zone is positioned along said flow path, said sample zone comprising a loading port for introducing said biological sample in liquid form into the flow path; wherein said reagent zone is positioned along the flow path downstream from said sample zone, said reagent zone comprising a first binding agent which is mobilizable by flow of said sample along said flow path, said first binding agent comprising a detectable label and being capable of specifically binding unfolded p53 protein to form a first complex; and wherein said detection zone is positioned downstream from said reagent zone along said flow path, and wherein flow of said sample along said flow path mobilizes said first binding agent to said detection zone; wherein said first binding agent comprises an antibody or fragment thereof comprising heavy chain CDRs 1-3 and light chain CDRs 1-3, wherein said heavy chain CDRs 1-3 comprise heavy chain CDR1(SEQ ID NO: 8), CDR2(SEQ ID NO: 9), and CDR3(SEQ ID NO: 10) and wherein said light chain CDRs 1-3 comprise light chain CDR1(SEQ ID NO:11), CDR2 (SEQ ID NO:12) and CDR3(SEQ ID NO: 13), and wherein formation of said first complex generates a detectable signal in said detection zone, indicating the presence of unfolded p53 protein in said biological sample at a concentration at or above 600±100 pg/ml.

In another aspect, the disclosure provides a device for detecting the presence of unfolded p53 protein in a biological sample, the device comprising: a test strip defining a flow path comprising a sample zone, a reagent zone, and capture zone; wherein said sample zone is positioned along said flow path, said sample zone comprising a loading port for introducing said sample in liquid form into said flow path; wherein said reagent zone is positioned downstream from said sample zone, said reagent zone comprising a first binding agent which is mobilizable by flow of said sample along said flow path, the first binding agent being capable of specifically binding unfolded p53 protein to form a first complex; wherein said capture zone is positioned downstream of said reagent zone along said flow path, said capture zone comprising an immobilized second binding agent comprising a detectable label capable of binding to and thereby immobilizing said first complex; wherein said first binding agent comprises an antibody or fragment thereof comprising heavy chain CDRs 1-3 and light chain CDRs 1-3, wherein said heavy chain CDRs 1-3 comprise heavy chain CDR1 (SEQ ID NO: 8), CDR2(SEQ ID NO: 9), CDR3(SEQ ID NO: 10) and wherein said light chain CDRs 1-3 comprise light chain CDR1(SEQ ID NO:11), CDR2(SEQ ID NO:12) and CDR3(SEQ ID NO: 13), and wherein immobilization of said first complex at said capture zone produces a detectable signal in said capture zone, said detectable signal indicating the presence of unfolded p53 protein in said biological sample at a concentration above 600±100 pg/ml.

In some embodiments of any of the aforesaid devices, further comprises a control zone comprising a secondary antibody that binds to the first binding agent to produce a detectable signal.

In some embodiments of any of the aforesaid devices, wherein said first binding agent comprises a heavy chain variable region and a light chain variable region, wherein said heavy chain comprises SEQ ID NO: 6 and said light chain comprises SEQ ID NO:7.

In some embodiments of any of the aforesaid devices, wherein said first binding agent comprises a heavy chain and a light chain, said heavy chain comprising SEQ ID NO: 4 and said light chain comprising SEQ ID NO: 5.

In some embodiments of any of the aforesaid devices, wherein said detectable label comprises one or more of colloidal gold particle, colloidal silver particle, colloidal platinum particle, latex beads, latex nanocomposites, magnetic beads. Quantum dots, fluorescent dyes, ruthenium complexes, nanocomposite particles and paramagnetic labels.

In some embodiments of any of the aforesaid devices, wherein said device further comprises an accelerant positioned alongside the longitudinal plane of the test strip that helps disperse the liquid sample across the flow path.

In some embodiments of any of the aforesaid devices, wherein said accelerant comprises a heating strip positioned alongside the longitudinal plane of the test strip effective to apply sufficient heat to the test strip so as to uniformly disperse liquid sample along the flow path.

In some embodiments of any of the aforesaid devices, wherein said accelerant comprises a magnetic strip positioned alongside the longitudinal plane of the test strip that applies magnetic field to ensure uniform dispersal of liquid sample across the flow path.

In some embodiments of any of the aforesaid devices, wherein said magnetic field is a constant magnetic field.

In some embodiments of any of the aforesaid devices, wherein said magnetic field is a dynamic magnetic field or a constant magnetic field.

In another aspect, the disclosure provides, a method for detecting the onset of dementia leading to Alzheimer's disease (AD) in a subject who does not exhibit one or more symptoms of dementia, the method comprising: contacting a biological sample from said subject with any of the devices of the invention by introducing said sample in liquid form into said loading port of said sample zone of said device; and observing said detection zone of said device for a signal; wherein visualization of a signal in said detection zone indicates the onset of dementia in said subject.

In another aspect, the disclosure provides a method for confirming the presence of cognitive decline due to dementia leading to AD in a subject who exhibits one or more symptoms of dementia, the method comprising: contacting a biological sample from said subject with any of the devices of the invention by introducing said sample in liquid form into said loading port of said sample zone of said device; and observing said detection zone of said device for a signal; wherein visualization of a signal in said detection zone confirms the presence of dementia in said subject.

In some embodiments of any of the aforesaid methods, wherein said observation of said signal in said detection zone indicates the presence of unfolded p53 protein in said biological sample at a concentration at or above 600±100 pg/ml.

In some embodiments of any of the aforesaid methods, wherein said symptoms comprise one or more of a cognitive and/or a psychological characteristic of progressive dementia.

In some embodiments of any of the aforesaid methods, wherein a said cognitive change comprises memory loss, diminution in one's ability to communicate, diminution in one's visual and spatial abilities, diminution in one's ability to reason and problem solve, diminution in one's ability to handle complex tasks, to plan, to organize, to coordinate complex tasks, to perform motor functions when compared with an individual who has normal levels of unfolded p53 protein.

In some embodiments of any of the aforesaid methods, wherein said psychological change comprises one or more of depression, anxiety, paranoia, agitation, and hallucinations.

In some embodiments of any of the aforesaid methods, wherein said subject has a family history of dementia.

In some embodiments of any of the aforesaid methods, wherein said subject is genetically predisposed to have dementia.

In some embodiments of any of the aforesaid methods, wherein said subject has abnormal expression of or has a genetic alteration that affects the function of one or more genes selected from the group consisting of APOE4, ABCA7, CLU, CR1, PICALM, PLD3, TREM2, SORL1, APP, PSEN1 and PSEN2.

In some embodiments of any of the aforesaid methods, wherein said sample is a blood sample.

In some embodiments of any of the aforesaid devices, wherein said sample is a blood sample.

In some embodiments of any of the aforesaid devices, wherein said first binding agent is an antibody able to detect unfolded p53 protein having a sequence shown in SEQ ID NO:3 and said variant being in a concentration greater than 600±100 pg/ml in the liquid sample.

In some embodiments of any of the aforesaid devices, wherein said first binding agent is able to bind unfolded p53 having one of more post translational modifications (PTM) in the region of amino acids 1-137 of the p53 protein.

In some embodiments of any of the aforesaid devices, wherein said PTMs are selected from the group consisting of: PTM-1 at the amino acid M1, PTM-2 at the amino acid K164, PTM-3 at the amino acid K370, PTM-4 at the amino acid L101, PTM-5 at the amino acid K120, PTM-6 at the amino acid K132, PTM-7 at the amino acid K139, PTM-8 at the amino acid K291, PTM-9 at the amino acid K357, PTM-10 at the amino acid S6, and PTM-11 at the amino acid S33.

In some embodiments of any of the aforesaid devices, wherein said secondary antibody is anti-IgM antibody.

In another aspect, the disclosure provides a device comprising a macroporous body containing in the dry state a labelled specific binding reagent such as anti-U-p53 antibody that is freely soluble or dispersible in an aqueous sample.

In another aspect, the disclosure provides a device comprising a macroporous body containing in the dry state a labelled specific binding reagent comprising an antibody or fragment thereof comprising heavy chain CDRs 1-3 and light chain CDRs 1-3, wherein said heavy chain CDRs 1-3 comprise heavy chain CDR1 (SEQ ID NO: 8), CDR2(SEQ ID NO: 9), and CDR3(SEQ ID NO: 10) and wherein said light chain CDRs 1-3 comprise light chain CDR1(SEQ ID NO: 11), CDR2 (SEQ ID NO:12) and CDR3(SEQ ID NO: 13), wherein said labelled specific binding reagent is freely soluble or dispersible upon contact with an aqueous sample.

In another aspect, the disclosure provides a method of detecting unfolded p53 variant (U-p53^{AZ}) in a biological sample of a subject comprising the steps of: contacting said biological sample of said subject with any of the aforesaid devices by introducing the sample in liquid form to the loading port of the sample zone and observing the detection zone for the presence of absence of a signal, wherein the presence of a signal in the detection zone indicates the presence of U-p53AZ in said sample, and the absence of a signal in the detection zone indicates the absence of U-p53AZ in the sample.

In some embodiments of any of the aforesaid methods, wherein said subject has no indicators of dementia.

In some embodiments of any of the aforesaid methods, wherein the presence of U-p53^{AZ} in said sample when said sample is from a subject who displays no indicators of dementia signifies an increased risk of developing progressive dementia leading to AD.

In some embodiments of any of the aforesaid methods, wherein said subject has two or more indicators of dementia.

In some embodiments of any of the aforesaid methods, wherein the presence of U-p53AZ in said sample when said sample is from a subject who displays five or more indicators of dementia signifies progressive dementia leading to AD.

In some embodiments of any of the aforesaid methods, wherein indicators of dementia include hallucination, memory loss, depression, paranoia and aggressive behavior.

In some embodiments of any of the aforesaid methods, wherein said five indicators of dementia are selected from the group comprising: memory loss, hallucination, paranoia, aggressive behavior, depression, difficulty communicating, difficulty with visual and spatial abilities, difficulty with reasoning and problem-solving, difficulty handling complex tasks, difficulty with planning and organizing, difficulty with coordination and motor functions, agitation, confusion and disorientation.

In some embodiments of any of the aforesaid methods, wherein said subject has an increased risk of having dementia within the next 10 years.

In some embodiments of any of the aforesaid methods, wherein said subject has a lower risk of having dementia within the next year.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A shows an isometric view of an example of the assay device. FIG. 1B. is a representative view of the test device. FIG. 1C shows a cross sectional view of the device shown in FIG. 1A. FIG. 1D shows an example of a schematic of lateral flow assay to detect the presence of analyte (U-p53^{AZ}) in sample using an antibody (2D3A8). An exemplary conventional lateral flow device is illustrated in FIGS. 1A, 1B & 1C.

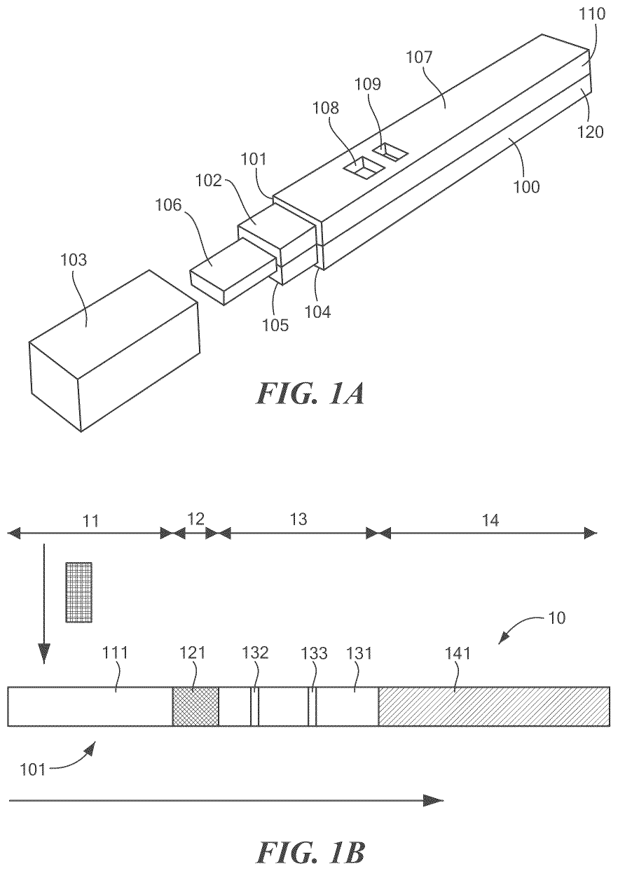

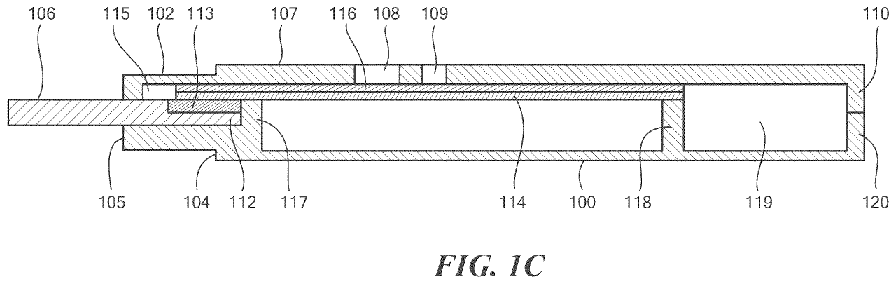

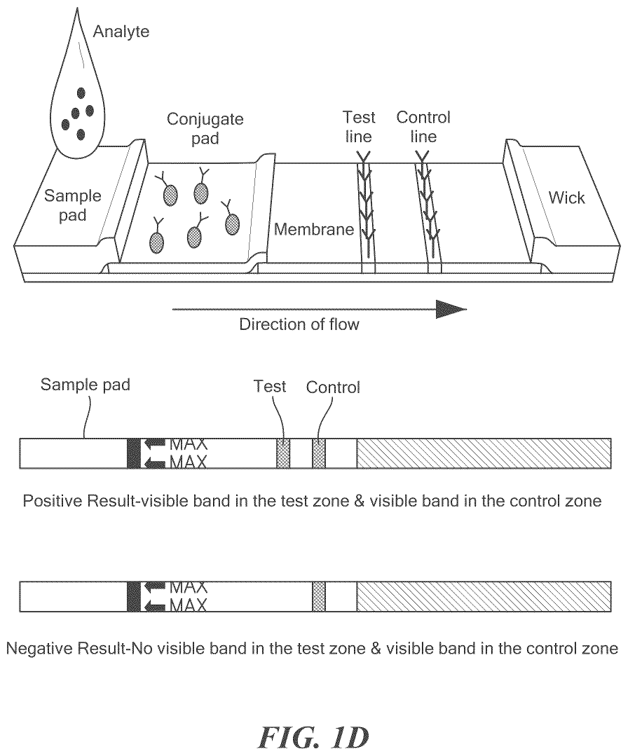

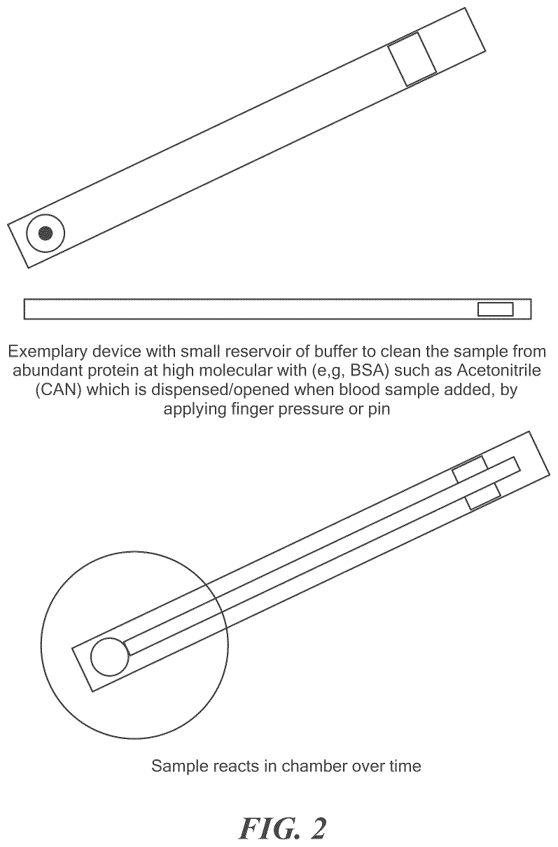

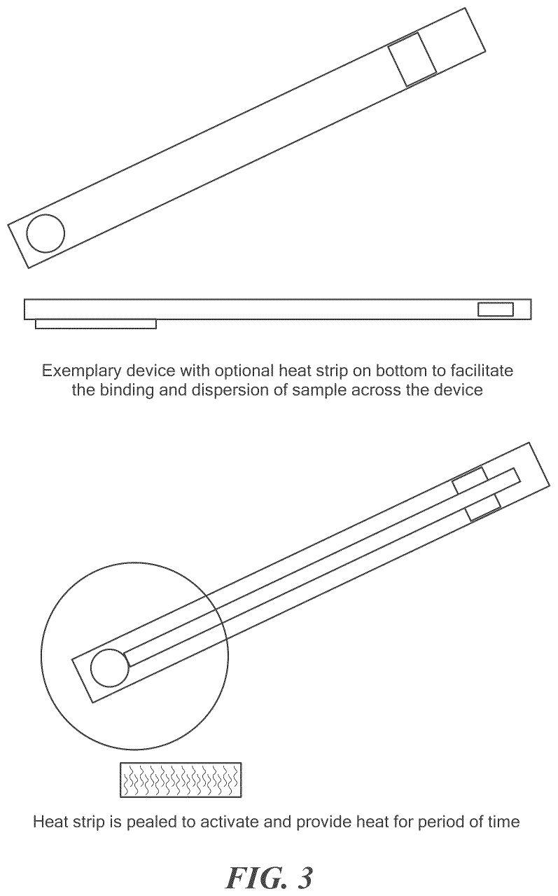

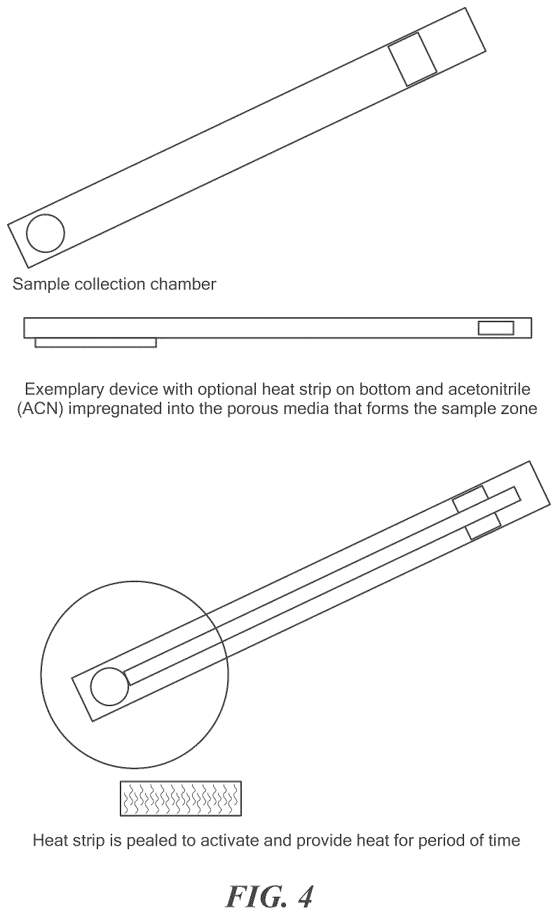

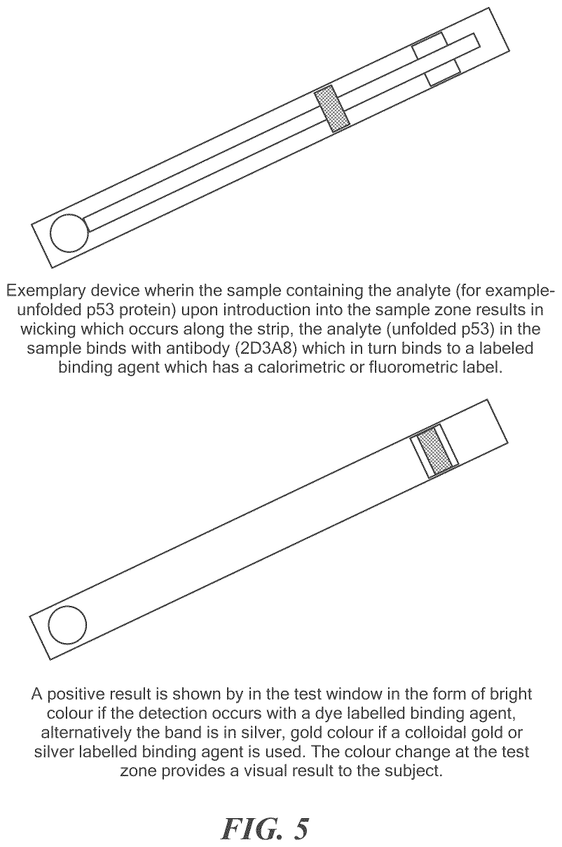

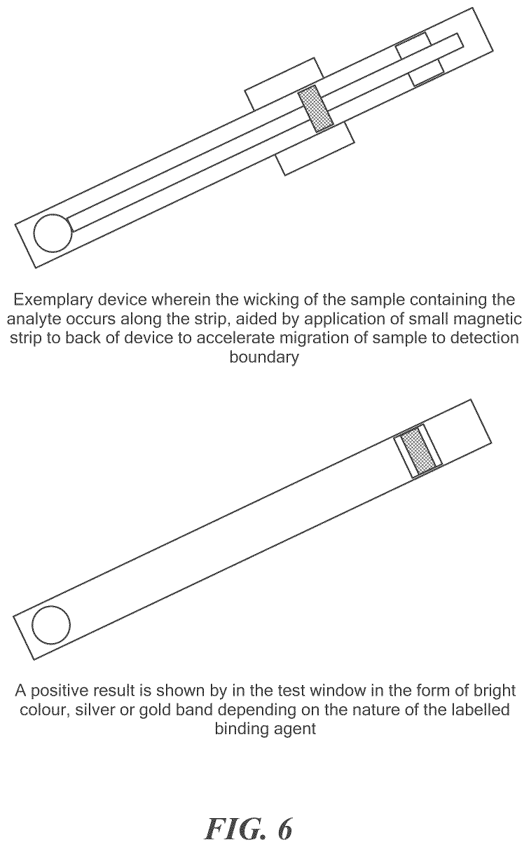

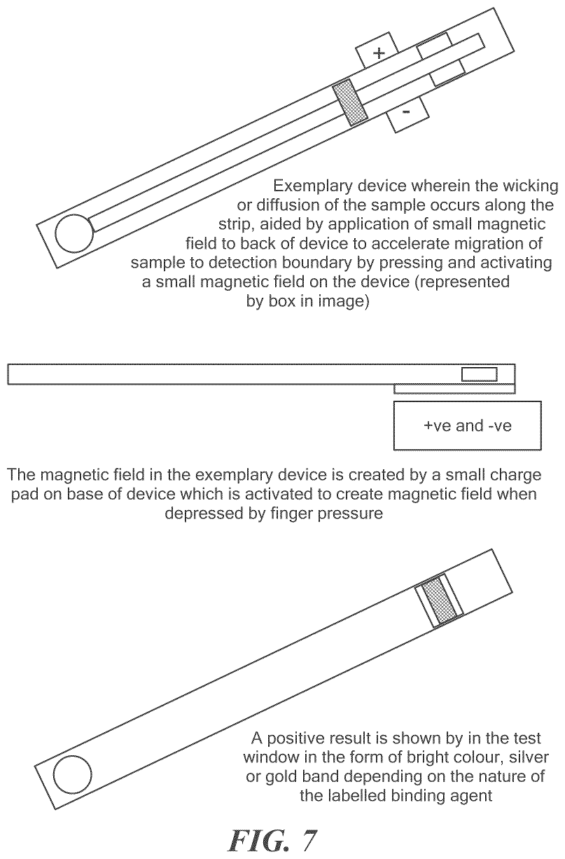

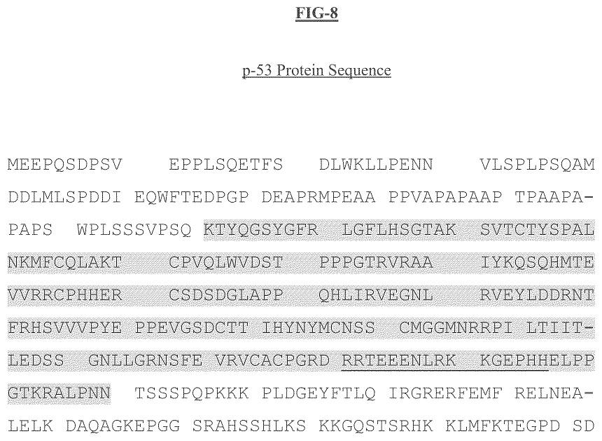

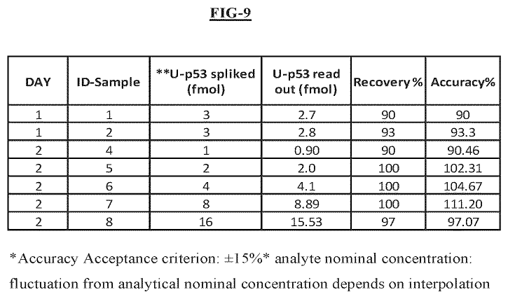

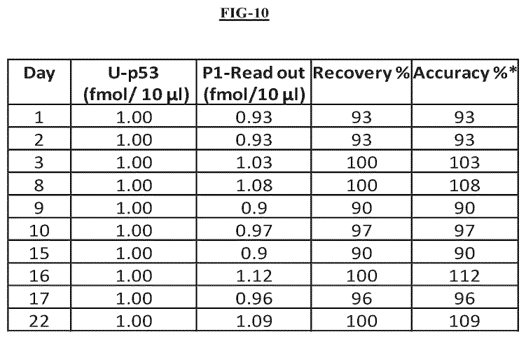

The device of FIG. 1A comprises a housing or casing 100 of elongate rectangular form having at one end 101 a portion 102 of reduced cross-sectional area. A cap 103 can be fitted onto portion 102 and can abut against the shoulder 104 at end 101 of the housing. Cap 103 is shown separated from housing 100. Extending beyond end 105 of portion 102 is a porous sample collector 106. When cap 103 is fitted onto portion 102 of the housing, it covers porous sample collector 106. Upper face 107 of housing 100 incorporates two apertures 108 and 109. The housing is constructed of an upper half 110 and a lower half 120.

The lateral flow test device (10) of FIG. 1B typically includes a sample zone (11), a reagent zone (12) a test zone (13) and adsorbent zone (14). Typically, the sample receiving zone (11) contains a porous sample receiving pad 111 and the reagent zone (12) contains a conjugate pad 121 where all necessary assay reagents are contained therein. The test zone (13) comprises a test strip (131) with a test line (132) and a control zone with a control line (133) is located downstream. A colorimetric or visual readout appearing in the test result zone typically indicates the presence of the analyte being tested. The lateral flow device typically contains an absorbent pad 141, which is in fluid communication with the elements 111, 121, and 131 along the direction indicated by the arrow of FIG. 1B.

The sample containing the analyte is deposited on the sample zone and it migrates towards the reagent zone containing the first antibody that is labeled with a detectable moiety such as gold nanoparticles or latex particles. The analyte in the sample is bound by the antibody forming a complex which then migrates further into the test zone. The complex then bind with the second antibody to generate a visible signal in the test zone. In some embodiments, the detectable label can be on the second antibody and not on the first antibody, in these embodiments the complex becomes visible only after binding with second antibody in the test zone. In some embodiments, a control zone is optionally present where a visible signal is generated when the first antibody containing the detectable label is able to migrate to the control zone serving as a control. The FIG. 1B also shows schematic examples of a positive result indicating two visible band one for the test zone and another for the control zone. Likewise, in the negative result schematic, only the control zone shows a visible band confirming the fidelity of the reagents in the device.

FIG. 2 is an illustration of an embodiment of the test device having small reservoir of reagent for protein high molecular weight precipitation such as Acetonitrile (ACN) in the sample zone which is dispensed/opened when blood sample added, by applying finger pressure or pin. In an alternate embodiment, the Acetonitrile impregnated into the porous media that forms the sample zone.

FIG. 3 is an illustration of an embodiment of the test device having an optional heat strip on bottom to facilitate the binding and dispersion of sample across the device.

FIG. 4 is an illustration of an embodiment of the test device having an optional heat strip on bottom and acetonitrile (ACN) impregnated into the porous media that forms the sample zone.

FIG. 5 is an illustration of an embodiment of the test device wherein the sample containing the analyte upon introduction into the sample zone results in wicking which occurs along the strip, the analyte in the sample binds with antibody which in turn binds to a labeled binding agent which has a calorimetric or fluorometric label.

FIG. 6 is an illustration of an embodiment of the test device wherein the wicking of the sample containing the analyte occurs along the strip, aided by application of small magnetic strip to back of device to accelerate migration of sample to the detection boundary.

FIG. 7 is an illustration of an embodiment of the test device wherein the wicking of the sample occurs along the strip, aided by application of small magnetic field to back of device to accelerate migration of sample to detection boundary by pressing and activating a small magnetic field on the device. The magnetic field can be either constant magnetic field or the magnetic field can be turned on or off by means of a switch.

FIG. 8 discloses p53 protein sequence (SEQ ID NO: 3), the DNA binding domain of p53 protein is highlighted in grey, and the linear epitope (SEQ ID NO: 1) recognized by 2D3A8 antibody is underlined.

FIG. 9 discloses the Unfolded p53^{AZ} (by Nitrosilation reaction) recovery percentages of human plasma samples using 2D3A8 conjugated beads.

FIG. 10 discloses the Unfolded p53^{AZ} (by Nitrosilation reaction) recovery percentages of human plasma samples using 2D3A8 conjugated beads over the course of several days post conjugation.

### DETAILED DESCRIPTION OF INVENTION

The presence of an altered isoform of the p-53 protein in subjects with a cognitive decline due to a neurogenerative disease such as AD, MCI are disclosed in WO2016050630 and WO2020178620. WO2016050630 discloses an anti-human p53 antibody (2D3A8) that specifically binds to a linear epitope found only in a conformationally altered variant of p53 protein of subjects with Alzheimer's disease or prone to develop Alzheimer's disease or cognitive impairment during ageing. The ability of the 2D3A8 monoclonal antibody to specifically bind to and detect unfolded p53 (U-p53^{AZ}) is also disclosed in aforesaid publications, the contents of each of which are herein incorporated by reference in their entirety.

Neurological disorders such as dementia, Parkinson's disease, Alzheimer's disease and multiple sclerosis are often observed and/or diagnosed in a subject by differential diagnosis including medical history, present symptoms, physical finding, a neuropsychological test, a blood test, imaging test, and the like. Frequently, the presence of a neurological disorder is diagnosed at an advanced stage where significant damage such as accumulation of prions or loss of memory, and neurological damage has already occurred. These medical tests require consultation with doctors and typically involve multiple in person visits to various labs involving expensive procedures such as CT, PET, MRI scans and extensive blood work. The definitive diagnosis of the presence of a neurological disorder often takes months. Currently, there exists no device or test that would provide a result in a short amount of time and indicate the presence or absence of a neurological disorder from a simple at-home blood test that uses one or more drops of biological fluid such as blood.

It would therefore be beneficial for a subject to be able to test for the presence or onset of dementia leading to AD, which is a neurological disorder, from the comfort of the subject's homes or in a medical provider's office by means of a device to which a biological sample, from 1 to 3 drops, of said subject is applied. The invention provides a device which allows for rapid testing of a blood sample in a non-laboratory setting and produces a rapid result indicating the presence of dementia leading to AD and/or predicts onset of dementia leading to AD in a subject.

The invention also encompasses a macroporous body containing in the dry state a labelled specific binding reagent such as anti-unfolded-p53 antibody that is freely soluble or dispersible in an aqueous sample that may be applied to the macroporous body. The invention further encompasses any analytical device that incorporates such a macroporous body together with a test strip or the like into which liquid sample carrying dissolved or dispersed labelled specific binding reagent can flow from the macroporous body.

The present invention relates to assays involving specific binding, especially immunoassays. In particular, the invention relates to analytical devices which are suitable for use in the home, clinic or doctor's surgery and which are intended to give an analytical result rapidly and which require the minimum degree of skill and involvement from the user. The analytical result can be observable within a matter of minutes following sample application, e.g. thirty minutes or less.

The device can also be used a physician or a physician assistant who can administer the test as way to triage a subject for diagnosis of dementia leading to AD before requiring extensive tests such as PET or MRI scans which are significantly more expensive.

For instance, a subject having no symptoms of dementia can undertake the test using the device of the invention to determine whether he or she has progressive dementia leading to AD which is at a stage that has not yet manifested as symptoms discernable by the subject.

In another example, a subject having a family history of dementia such as a sibling, a parent, and/or a grandparent who has or have suffered from can undertake the test using the device of the invention to determine whether the subject is at risk of developing the neurological disorder within the next 6-10 years.

In some embodiments, a subject having a family history of dementia such as a sibling, a parent, and/or a grandparent who has or have suffered from can undertake the test using the device of the invention to determine whether the subject is at risk of developing the neurological disorder within the next 18 months-10 years.

In another example, a subject having one or more symptoms of an early stage of dementia, such as memory loss, disorientation, hallucination, etc., can undertake the test using the device of the invention to determine whether he or she has dementia leading to AD and therefore can seek treatment with a physician after a positive test as indicated by the presence of a visible signal on the device. In such a case, if the device produces a negative result, indicating that the concentration of U-p53^{AZ} is lower than the threshold of about 500 pg/ml, it signals that the subject is not at risk for developing the neurological disorder within 6-10 years. Optionally, the test can be repeated by such subjects after a time such as a year or two to ensure that the risk has not progressed.

In some embodiments, the concentration of unfolded p53 (for example U-p53^{AZ}) in the sample of a subject is at least 600±100 pg/ml, said sample when tested on the device of the invention will generate a positive signal since the concentration of unfolded p53 is above the threshold of detection.

In some embodiments, the concentration of unfolded p53 (for example U-p53AZ) in the sample of a subject is at least 700 pg/ml, said sample when tested on the device of the invention will generate a positive signal since the concentration of unfolded p53 is above the threshold of detection. Each value noted here includes a variation of +/−25, +/−50, +/−100 pg/ml from the stated amount. For instance, the concentration of unfolded p53 sample may range from one of 600 pg/ml, 650 pg/ml, 675 pg/ml, 700 pg/ml, 725 pg/ml, 750 pg/ml, and 800 pg/ml.

In some embodiments, the concentration of unfolded p53 (for example U-p53^{AZ}) in the sample of a subject is at least 800 pg/ml, said sample when tested on the device of the invention will generate a positive signal since the concentration of unfolded p53 is above the threshold of detection. Each value noted here includes a variation of +/−25, +/−50, +/−100 pg/ml from the stated amount. For instance, the concentration of unfolded p53 sample may range from one of 700 pg/ml, 750 pg/ml, 775 pg/ml, 800 pg/ml, 825 pg/ml, 850 pg/ml, and 900 pg/ml.

In some embodiments, the concentration of unfolded p53 (for example U-p53^{AZ}) in the sample of a subject is at least 900 pg/ml. Each value noted here includes a variation of +/−25, +/−50, +/−100 pg/ml from the stated amount. For instance, the concentration of unfolded p53 sample may range from one of 800 pg/ml, 850 pg/ml, 875 pg/ml, 900 pg/ml, 925 pg/ml, 950 pg/ml, and 1000 pg/ml.

In some embodiments, the concentration of unfolded p53 (for example U-p53^{AZ}) in the sample of a subject is at least 1000 pg/ml. Each value noted here includes a variation of +/−25, +/−50, +/−100 pg/ml from the stated amount. For instance, the concentration of unfolded p53 sample may range from one of 900 pg/ml, 950 pg/ml, 975 pg/ml, 1000 pg/ml, 1025 pg/ml, 1050 pg/ml, and 1100 pg/ml.

In some embodiments, the concentration of unfolded p53 (for example U-p53^{AZ}) in the sample of a subject is between 600 to 1000 pg/ml with variations of +/−25, +/−50, +/−100 pg/ml from the stated amount.

In some embodiments a subject having no symptoms of dementia and have tested negative using the device of the invention (i.e. no signal detected when the sample from subject is tested using the device) indicating that the levels of unfolded p53 in the sample from said subject are below the detection limit and such subject may yet develop progressive dementia leading to AD during the follow up period of up to 10 years. It is therefore advantageous for those subjects to repeat the test every year to ensure that the levels of unfolded p53 protein remain low and are below the threshold of detection of the device.

In some embodiments, a subject having no symptoms of dementia and have tested negative using the device of the invention, is tested again after a follow up period of 1 year to see if there is onset of dementia leading to AD.

In some embodiments, a subject having no symptoms of dementia and have tested negative using the device of the invention, is tested again after a follow up period of 2 years for predicting the onset of dementia leading to AD.

In some embodiments, a subject having no symptoms of dementia and have tested negative using the device of the invention, is tested again after a follow up period of 3 years for predicting the onset of dementia leading to AD.

In some embodiments, a subject having no symptoms of dementia and have tested negative using the device of the invention, is tested again after a follow up period of 4 years for predicting the onset of dementia leading to AD.

In some embodiments, a subject having no symptoms of dementia and have tested negative using the device of the invention, is tested again after a follow up period of 5 years for predicting the onset of dementia leading to AD.

In some embodiments, a subject having no symptoms of dementia and have tested negative using the device of the invention, is tested again after a follow up period of 6 years for predicting the onset of dementia leading to AD.

In some embodiments, a subject having no symptoms of dementia and have tested negative using the device of the invention, is tested again after a follow up period of 7 years for predicting the onset of dementia leading to AD.

In some embodiments, a subject having no symptoms of dementia and have tested negative using the device of the invention, is tested again after a follow up period of 8 years for predicting the onset of dementia leading to AD.

In some embodiments, a subject having no symptoms of dementia and have tested negative using the device of the invention, is tested again after a follow up period of 9 years for predicting the onset of dementia leading to AD.

In some embodiments, a subject having no symptoms of dementia and have tested negative using the device of the invention, is tested again after a follow up period of up to 10 years for predicting the onset of dementia leading to AD.

In some embodiments, the unfolded p53 (for example U-p53^{AZ}) comprises one of more post translational modifications (PTM) in the region of amino acids 1-137 of the p53 protein. In some embodiments, the PTMs are selected from the group consisting of PTM-1 at the amino acid M1, PTM-2 at the amino acid K164, PTM-3 at the amino acid K370, PTM-4 at the amino acid L101, PTM-5 at the amino acid K120, PTM-6 at the amino acid K132, PTM-7 at the amino acid K139, PTM-8 at the amino acid K291, PTM-9 at the amino acid K357, PTM-10 at the amino acid S6, and PTM-11 at the amino acid S33.

The PCT application, PCT/IB2021/056792, describes in detail eleven main post-translation modifications (PTMs) in the amino acidic sequence of the p53 protein within the region of amino acids 1-371, herein called PTM-1, PTM-2, PTM-3, PTM-4, PTM-30 5, PTM-6, PTM-7, PTM-8, PTM-9, PTM-10, PTM-11 and/or some truncated forms of the p53 protein in a biofluid sample.

The PTM-1 is at the amino acid M1, the PTM-2 is at the amino acid K164, the PTM-3 is at the amino acid K370, the PTM-4 is at the amino acid L101, the PTM-5 is at the amino acid K120, the PTM-6 is at the amino acid K132, the PTM-7 is at the amino acid K139, the PTM-8 is at the amino acid K291, the PTM-9 is at the amino acid K357, the PTM-10 is at the amino acid S6, the PTM-11 is at the amino acid S33.

The post-translation modification PTM-1 has a group CO—CH3 branched to the amino acid M1 of the p53 protein; the post-translation modification PTM-2 has a group CO—CH3 branched to the amino acid K164 of the p53 protein; the post-translation modification PTM-3 has a group CO—CH3 branched to the amino acid K370 of the p53 protein; the post-translation modification PTM-4 has a ubiquitination site [GG] branched at the amino acid K101 of the p53 protein; the post-translation modification PTM-5 has a ubiquitination site [GG] branched at the amino acid K120 of the p53 protein, where [GG] denotes a lateral chain of two residues of “Glycine”; the post-translation modification PTM-6 has a ubiquitination site [GG] branched at the amino acid K132 of the p53 protein; the post-translation modification PTM-7 has a ubiquitination site [GG] branched at the amino acid K139 of the p53 protein; the post-translation modification PTM-8 has a ubiquitination site [GG] branched at the amino acid K291 of the p53 protein;—the post-translation modification PTM-9 has a ubiquitination site [GG] branched at the amino acid K357 of the p53 protein; the post-translation modification PTM-10 has phosphorylation at the amino acid S6 of the p53 protein; the post-translation modification PTM-11 has phosphorylation at the amino acid S33 of the p53 protein.

In some embodiments, the biological sample from the subject is tested using anyone of the devices of the invention to determine if the unfolded p53 protein (for example U-p53^{AZ}) is present in the sample. The sample from the subject is subjected to immunoprecipitation using anti-p53 antibody followed by protease digestion and mass spec analysis as described PCT/IB2021/056792 to determine the type of PTM that occur in the unfolded p53 protein of the sample.

Briefly, the sample from the subject was immunoprecipitated by using anti-p53 antibody, followed by protease digestion using trypsin. The digested sample was then analyzed by HPLC-mass spectrometry, Peptide Mass Fingerprint and Database Search. Samples extracted from the different categories of patients were thawed at room temperature under laminar flow cabinet for 30 min.

### Plasma Chemical Contaminants Depletion and Immune Precipitation

The sample was spiked in 25 μL aliquots. 5 μL of CH3CN were added to 25 μL of plasma. The sample centrifugation takes place at 13000 g for 10 minutes. 40 μL of supernatant was added to the bead-anti-p53-antibody complex. The mixture was incubated at room temperature for 1 hour and then at 4° overnight. A magnetic surface was used to remove the supernatant. 500 μl of Buffer A (Tris 25 mM, Sodium Chloride (NaCl) 0.15 mM, Tween-20 50 mM) was added to the mixture and was vortexed. A magnetic surface was used to remove the supernatant. 45 μl of Buffer B (Glycine 0.1 M pH 2.0) was added to the pellet and incubated for 10 minutes at room temperature. A magnetic surface is used to collect the eluate (40 μL).

### Enzymatic Digestion of the Immunocaptured p53 Protein

2.15 μl of Dithiothreitol (DTT) 180 mM were added to 40 μL of the eluate. The mixture was incubated for 15 min at 50° C. and at room temperature for 30 minutes; 2.15 μl of Iodoacetamide (IAA) 400 mM was added 42.15 μL of the mixture. The obtained mixture was incubated for 15 minutes at room temperature. 2.15 μL of AmBic 50 mM were added 44.30 μL of the obtained mixture. 1 μL of trypsin (25 ng/μL) containing Lys-c (50 ng/μL) and AmBic 50 mM was added to 46.45 μL of the obtained mixture. Incubation takes place at 37° C. for 3.5 hours followed by 57° C. for 30 minutes. 1 μL of Formic Acid (HCOOH) was added to 47.45 μL of the obtained mixture to stop the enzymatic digestion. pH value is checked, and it has to be in the range 1-4. If it is higher than 4 progressive volume (1 μL) of Formic Acid was added to obtain a pH value between 1 and 4. 10 μL of the obtained sample were analysed.

### Detection of PTMs by LC-SACI-MS

HPLC Ultimate 3000 (Thermofisher, USA) with a Phenomenex Kinetex PFP 50×4.1 mm 2.6 μm was used to perform the chromatographic analysis. Binary gradient was used: Phase A (H2O+0.2% Formic Acid (HCOOH)) and Phase C acetonitrile (CH3CN). The gradient was reported in the table below. 10 μL of sample was injected. 5 LTQ Orbitrap XL was used for the data acquisition. SACI ionization source was employed.

The potential surface was 47 V, Gas nebulizer pressure was 75 Psi and dry gas flow was 1.0 L/min. 350° C. of nebulizer temperature was employed together with 320° C. of dry gas one. SACI peptide adduct profile mode was employed for data acquisition (Cristoni et al. Rapid Commun Mass Spectrom. 2003;17(17):1973-81.). Protein sequence and PTM data is obtained using the SANIST-prot tool operating in bottom up conditions.

The plasma samples of 7 patients affected by AD, 5 cognitive unimpaired (CU), 2 patients affected by MCI, 6 frontal dementia (FD), 1 patient with vascular dementia (VD) and 1 patient with Lewy Body dementia (LB) and 6 patients with MCI to AD and 6 patients CU to AD have been treated with the experimental protocol based on protein L to isolate 20 protein p53 disclosed above.

The extracted and sequenced p53 protein from the different clinical groups in addition to a different linear sequence, corresponding accordingly to different molecular weight, also showed post-transductional modifications (PTMs), mainly characterized by ubiquitination, acetylation and phosphorylation on specific amino acid residues. The p53 protein extracted from subjects with AD showed truncation in the region of amino acid 1-248 with respect to the wt p53 protein.

Blood samples from Cognitive unimpaired subjects (CU) showed the presence of at least two PTMs selected from PTM-2, PTM-7, PTM-8, and PTM-11. Blood samples from subjects with neurological diseases show the presence of at least two PTMs selected from PTM-1, PTM-3, PTM-4, PTM-5, PTM-6, PTM-9, and PTM-10 and at least one PTM selected from PTM-2, PTM-7, PTM-8, and PTM-11.

The presence of at least two PTMs selected from PTM-4, PTM-5, and PTM-9 in the sample from a subject is indicative of an asymptomatic subject having the prognosis of cognitive decline of Alzheimer's dementia or dementia leading to Alzheimer's disease (AD). The presence of at least two PTMs selected from PTM-1, PTM-3, PTM-5, PTM-6, and PTM-10 in the sample from a subject is indicative of MCI with a prognosis of cognitive decline of AD. The presence of PTM-5, and PTM-9 in the sample from a subject is indicative of FTD. The presence of PTM-5, and PTM-6 in the sample from a subject is indicative of LB. The presence of PTM-4, and PTM-5 in the sample from a subject is indicative of VD.

The presence of different PTMs in subjects with different neurological diseases indicate that there are different types/isoforms of unfolded p-53 protein found in the sample of subject with different neurological diseases.

In some embodiments, the device of the invention generates a signal when the biological sample from a subject is applied on to the sample port of the device. The generation of a signal indicates that the concentration of U-p53^{AZ} is present in the sample. The absence of a signal indicates that the concentration of U-p53^{AZ} is absent in the sample.

In some embodiments, the device comprising a macroporous body containing in the dry state a labelled specific binding reagent such as anti-U-p53 antibody that is freely soluble or dispersible in an aqueous sample.

In some aspects, the disclosure provides a method of detecting unfolded p53 variant (U-p53^{AZ}) in a biological sample of a subject comprising the steps of: contacting said biological sample of said subject with any of the devices of the invention by introducing the sample in liquid form to the loading port of the sample zone and observing the detection zone for the presence of absence of a signal, wherein the presence of a signal in the detection zone indicates the presence of U-p53AZ in said sample, and the absence of a signal in the detection zone indicates the absence of U-p53AZ in the sample.

In some embodiments of any of the aforesaid methods, the subject has no indicators of dementia. In some embodiments of any of the aforesaid methods, the subject has at least one indicator of dementia. In some embodiments of any of the aforesaid methods, the subject has two or more indicators of dementia. In some embodiments of any of the aforesaid methods, the subject has two indicators of dementia. In some embodiments of any of the aforesaid methods, the subject has three indicators of dementia. In some embodiments of any of the aforesaid methods, the subject has four indicators of dementia. In some embodiments, the indicators of dementia include hallucination, memory loss, depression and paranoia.

In some embodiments, where the subject has two or more indicators of dementia and the sample from the same subject generates a signal in the device of the invention indicating the presence of U-p53^{AZ}, then the subject has an increased risk of having dementia within the next 10 years. In some embodiments, the subject having an increased risk of having dementia within the next 10 years is advised to undertake lifestyle changes. In some embodiments, the lifestyle changes include but not limited to losing weight, reducing inflammation and consuming a healthy diet followed by evaluation of sample of said subject after 3-6 months. The evaluation of second sample after the lifestyle change would determine whether the risk of having dementia has decreased which would be reflected by the absence of U-p53^{AZ} in the sample or whether the risk has stayed the same indicating that the life style changes have not been effective in lowering the amounts of U-p53^{AZ} in the sample.

In some embodiments, where the subject has no indicators of dementia and the sample from the same subject does not generate a signal in the device of the invention indicating the absence of U-p53AZ, then the subject has a lower risk of having dementia within the next year. In some embodiments, the subject having a lower risk of dementia is advised to undertake evaluation of sample of said subject after a year.

In some cases, the device can be used as a way to monitor whether a particular drug therapy or change in lifestyle such as weight loss or increased exercise is helpful or harmful to the progression of dementia leading to AD. It is expected that if the concentration of U-p53^{AZ} increases over the threshold of 600±100 pg/ml in the subject biological sample, this will produce a positive signal in the test zone of the device, in which case it may be that a given drug therapy or change in lifestyle will not be useful or enough for preventing the progression of dementia. Similarly, if the concentration of U-p53^{AZ} has decreased below a threshold of about 600±100 pg/ml in the subject biological sample, this will produce a negative result in the test zone of the device, in which case it may be that a given drug therapy or change in lifestyle is useful for preventing the progression of dementia.

In some aspects, the disclosure provides a device that allows subjects to manage the progression of dementia leading to AD by testing their blood for the presence and/or increase in the amounts of U-p53^{AZ} protein. Dementia in many instances cannot be prevented but its progress can be potentially slowed down by undertaking changes in lifestyle such as reducing stress, avoiding excessive smoking and/or alcohol consumption, controlling inflammation, improving quality of sleep, maintaining healthy cholesterol, avoiding insulin fluctuations and consuming a healthy diet. The device can be used by the subject to determine if the levels of the conformational variant of -p-53protein, relevant in AD and preferentially identified as U-p53^{AZ} increase or decrease or is stable over time as a result of these active steps taken by the subject. Hence the subject can use the device as a feedback process to determine if their condition is improving or deteriorating with respect to the likelihood of worsening of dementia leading to AD to assess the effectiveness of the lifestyle changes undertaken by them.

In some aspects, the disclosure provides a device comprising a test strip defining a flow path, comprising: a sample zone positioned along the flow path, the sample zone comprising a loading port for introducing the liquid sample into the flow path; a reagent zone located downstream from the sample zone, said reagent zone comprising a first binding agent being mobilizable by the liquid sample, the first binding agent comprising a detectable label and being capable of binding to the U-p53^{AZ} protein to form a first complex; a test zone located downstream of the reagent zone along the flow path, and wherein formation of said first complex generates a detectable signal in the test zone indicating the presence of the conformational variant of p53 protein (U-p53^{AZ}) thereby predicting the onset of dementia leading to AD.

In some embodiments where the label attached to the binding agent is a particulate label such as latex beads, the macroporous body preferably has a pore size at least 10 times greater than the maximum particle size of the particulate label, to enable the particulate label to migrate freely out of the macroporous body with the liquid sample. The macroporous body comprises plastics material having an average pore size of not less than 10 microns, and ideally about 100 microns, because such larger pore sizes give better release of the labelled reagent. The plastics material should not be protein-binding or should be easily blockable by means of reagents such as BSA or PVA, to minimize non-specific binding and to facilitate free movement of the labelled reagent after the macroporous body has become moistened with the liquid sample. The plastics material can be pre-treated with surface active agent or solvent, if necessary, to render it more hydrophilic and to promote rapid uptake of the liquid sample. Alternatively, if desired, a surface-active agent can be incorporated in the solution containing the labelled reagent when this is applied to the macroporous material during manufacture of the device. The labelled reagent is preferably incorporated in the macroporous material in bulk, e.g., large sheet, form before it is subdivided into individual bodies for use in a testing device of the invention.

By incorporating the labelled reagent in a separate macroporous body, enhanced sensitivity is achieved because a substantial quantity of the liquid sample is able to take up the labelled reagent before migrating through the carrier material to the detection zone, enhancing potential reaction time without significantly increasing overall test time. Also, the liquid which permeates the carrier is of a more uniform and consistent composition.

The invention also provides an analytical method in which a device as set forth above is contacted with an aqueous liquid sample such as blood suspected of containing U-p53^{AZ}, such that the sample permeates by capillary action via the macroporous body through the porous solid carrier into the detection zone and the labelled reagent migrates therewith to the detection zone, the presence of analyte in the sample being determined by observing the extent (if any) to which the labelled reagent becomes bound in the detection zone.

In some embodiments, the labelled reagent is a specific binding reagent such as anti-Unfolded-p53 antibody for an analyte such as U-p53^{AZ}. The labelled binding reagent, the analyte (if present) and the immobilized unlabeled specific binding reagent cooperate together in a “sandwich” reaction. This results in the labelled reagent being bound in the detection zone if analyte is present in the sample. The two binding reagents must have specificities for different epitopes on the analyte.

In a further embodiment, the porous carrier is linked via the macro-porous body to a porous receiving member to which the liquid sample can be applied and from which the sample can permeate into the porous carrier. Preferably, the porous carrier and the macroporous body are contained within a moisture-impermeable casing or housing and the porous receiving member extends out of the housing and can act as a means for permitting a liquid sample to enter the housing and reach the porous carrier. The housing should be provided with means, e.g. appropriately placed apertures, which enable the detection zone of the porous solid phase carrier material (carrying the immobilized unlabeled specific binding reagent) to be observable from outside the housing so that the result of the assay can be observed. If desired, the housing may also be provided with further means which enable a further zone of the porous solid phase carrier material to be observed from outside the housing and which further zone incorporates one or more control reagents which enable an indication to be given as to whether the assay procedure has been completed.

In some embodiments, the device comprises a test strip defining a flow path, comprising: a sample zone positioned along the flow path, the sample zone comprising a loading port for introducing the liquid sample into the flow path; a reagent zone located downstream from the sample zone, said reagent zone comprising a first binding agent being mobilizable by the liquid sample, the first binding agent comprising a detectable label and being capable of binding to the U-p53^{AZ} protein to form a first complex; a test zone located downstream of the reagent zone along the flow path, the test zone comprising a second binding agent capable of binding with the first complex to form a second complex, and wherein formation of said second complex generates a detectable signal indicating the presence of the U-p53^{AZ} protein thereby predicting the onset of neurological disorders such as dementia leading to AD.

In some embodiments, the device optionally comprises a control zone comprising a control antibody that binds with the first binding agent to generate a signal serving as a positive control for the fidelity of the device and its reagents.

In some aspects, the disclosure provides a device and a method for detecting a low concentration of analyte such as U-p53^{AZ} protein in a sample fluid with high sensitivity and high accuracy. The device and methods of the present invention retain the specificity for detecting an analyte in a sample fluid.

In some embodiments, the devices comprises an effective amount of an antibody that is sufficient to bind p53 protein in an unfolded or misfolded conformation (conformational variant). 2D3A8 is an exemplary antibody that binds p53 protein in a misfolded conformation, specifically the 2D3A8 antibody is able to bind U-p53^{AZ}, the details of which are described in WO2016050630 and WO2020178620. The amount of antibody or 2D3A8 antibody in the device is sufficient to detect unfolded p53 protein (preferentially U-p53^{AZ}) present in the biological sample at a concentration of 500 picogram/ml or higher.

In some embodiments, the device is capable of detecting unfolded p53 protein (preferentially U-p53^{AZ}) present in the biological sample at a concentration greater than 500 picogram/ml.

In some embodiments, the device is capable of detecting unfolded p53 protein (preferentially U-p53^{AZ}) present in the biological sample at a range of 500-10,000 picogram/ml.

In some embodiments, the device is capable of detecting unfolded p53 protein (preferentially U-p53^{AZ}) present in the biological sample at a range of 500-9,000 picogram/ml.

In some embodiments, the device is capable of detecting unfolded p53 protein (preferentially U-p53^{AZ}) present in the biological sample at a range of 500-6,000 picogram/ml.

In some embodiments, the device is capable of detecting unfolded p53 protein, preferentially U-p53^{AZ} present in the biological sample at a range of 500-2000 picogram/ml.

In some embodiments, the preferred chromatographic material is nitrocellulose. Another example of chromatographic material is glass fiber filter paper. Another example of chromatographic material are nitrocellulose laminated strips which can be used in the test devices. When nitrocellulose is used, the material of the application pad is chosen based on its ability to premix the test sample and the first reagent, i.e., fluid-flow through a nitrocellulose membrane is laminar and does not provide the more turbulent flow characteristics which allows the initial mixing of test sample and application pad reagents within the chromatographic material. If nitrocellulose is used as the chromatographic material, then Porex® hydrophilic polyethylene frit or glass fiber filter paper shall be used as appropriate application pad materials because they enable the mixing and reaction of the test sample and application pad reagents within the application pad and before transfer to the chromatographic material.

In some embodiments, binding reagents are labelled with colloidal particles such as gold and selenium may be subjected to rapid chromatographic solvent transport on chromatographic media by means of selected solvents and chromatographic transport facilitating agents. Where the labelled material is a colloidal particle labelled material, an important advantage is provided by the present invention in that the binding affinity of the immobilized reagent may be such that it is capable of capturing a labelled component in the flowing chromatographic stream in such a way that the labelled binding component is accumulated in the capture zone and is clearly discernable over the background stream of non-concentrated colloidal particle labelled material.

In some embodiments, the colloidal particle labelled binding reagents are chromatographically mobile and capable of producing visually detectable signals. In some embodiments, the binding reagents labeled with colloidal particle or latex particle or latex composite nanoparticles are dried onto a chromatographic medium in the presence of a meta-soluble protein and are capable of being rapidly resolubilized in the presence of an appropriate solvent such as the sample or a chromatographic transport solvent.

In some embodiments, binding reagents are labelled with nanoparticle—latex nanocomposite particles made of noble metals such as gold or platinum.

In some embodiments, binding reagents are labelled with latex beads tagged with detector reagents such as colored or fluorescent dyes or quantum dots or ruthenium complexes.

In some embodiments, binding reagents are labelled with carbon nanoparticles, or enzyme labels or paramagnetic labels.

In some embodiments, the device comprises a small reservoir of Acetonitrile (CAN) in the sample zone which is released when a blood sample is added, by applying finger pressure or pin

In some embodiments, the solvent acetonitrile is impregnated into the chromatographic medium of the sample zone.

In some embodiments, the device comprises an optional heat strip on bottom to facilitate the binding and dispersion of sample across the device.

In some embodiments, the device comprises an optional heat strip on bottom and acetonitrile (ACN) impregnated into the porous media that forms the sample zone

In some embodiments of the device, the sample containing the analyte (U-p53^{AZ}) upon introduction into the sample zone results in wicking which occurs along the strip, the analyte in the sample binds with antibody which in turn binds to a labeled binding agent which has a calorimetric or fluorometric label.

In some embodiments of the device, the wicking of the sample containing the detect unfolded p53 protein (preferentially U-p53^{AZ}) occurs along the strip is aided by the application of small magnetic strip to back of device to accelerate migration of sample to detection boundary. In some embodiments of the device, the wicking of the sample occurs along the strip, aided by application of small magnetic field to back of device to accelerate migration of sample to detection boundary by pressing and activating a small magnetic field on the device. The magnetic field can be either constant magnetic field or the controlled magnetic field can be turned on or off by means of a switch. In some embodiments, the user of the device can apply the magnetic field by turning on a switch connected to the device. in a lateral flow device] In some embodiments, the user of the device can apply magnetic field by removing an adhesive strip that covers the magnetic surface. (See Wu et al., Magnetic-Nanosensor-Based Virus and Pathogen Detection Strategies before and during COVID-19, ACS Appl. Nano Mater. 2020, 3, 10, 9560-9580; Moyano et al. “Magnetic Lateral Flow Immunoassays.” Diagnostics (Basel, Switzerland) vol. 10,5 288. 8 May 2020; Liu et al., Ultrasensitive and Highly Specific Lateral Flow Assays for Point-of-Care Diagnosis, ACS Nano 2021, 15, 3, 3593-3611 Feb. 19, 2021).

In some embodiments, signal detection is facilitated by the movement of U-p53^{AZ} and anti-p53 complex under the application of magnetic field. For instance, the anti-p53 antibody is labeled with a magnetic bead and upon binding to U-p53^{AZ} it becomes mobile under the application of magnetic field.

In some embodiments, signal detection is facilitated by the movement of colored U-p53AZ and anti-p53 complex past a boundary marker. For instance, the anti-p53 antibody is labeled with a colored latex particle and upon binding to U-p53^{AZ} it becomes mobile under the application of magnetic field. In some embodiments, the signal detection is facilitated by the collection of colored U-p53^{AZ} and anti-p53 complex in a well or pond at the end of the adsorbent pad allowing the accumulation of the bound complex intensifying the color.

### Definitions

“Assaying” denotes testing for or detecting the presence of a substance or material, such as, but not limited to, a chemical, an organic compound, an inorganic compound, a metabolic product, a drug or a drug metabolite, an organism or a metabolite of such an organism, a nucleic acid, a protein, or a combination thereof. Optionally, assaying denotes measuring the amount of the substance or material. Assaying further denotes an immunological test, a chemical test, an enzymatic test, and the like.

“Analyte” as used herein refer to molecules that can be detected using the devices and methods of the invention. In some embodiments, the analyte is a molecule that can be suspended or dissolved in a liquid. These include molecules such as nucleic acids, amino acids, lipids, saccharides, hormones, proteins, drugs, drugs of abuse, biological warfare agents, toxins, vitamins, steroids, pesticides, industrial chemicals, analogs, derivatives, and metabolites thereof. Preferably, the analyte is unfolded p53 protein in a blood sample.

“Sample” as used herein refers to a biological sample in liquid form, such as blood, urine, plasma, serum. A liquid sample is a biological fluid of a subject; for example, blood, urine, plasma, serum.

“Blood” as used herein refers to fluid that circulates in the heart, arteries, capillaries, and veins of a vertebrate animal carrying nourishment. The term blood also encompasses derivatives of blood such as serum and plasma.

Devices as described herein can be used for the detection of unfolded p53 protein (preferentially U-p53^{AZ}) in samples. In particular, the devices of the invention can be used to determine the presence, absence, or concentration of the unfolded p53 protein (preferentially U-p53^{AZ}) using both competitive and non-competitive lateral flow assays. In some embodiments of the invention, the sample can be blood sample and the analyte is unfolded p53 protein (preferentially U-p53^{AZ}). An exemplary conventional lateral flow device is illustrated in FIG. 1B. The lateral flow test device (10) typically includes a sample zone (11), a reagent zone (12) and a test zone (13). Typically, the sample receiving zone (11) contains a porous sample receiving pad 111 and the reagent zone (12) contains a conjugate pad 121 where all necessary assay reagents are contained therein. The test zone (13) comprise a test strip (131) with a test line (132) and a control zone with a control line (133) is located downstream. A calorimetric or visual readout appearing in the test result zone typically indicates the presence of unfolded p53 protein (preferentially U-p53^{AZ}) being tested. The lateral flow device typically contains an absorbent pad 141, which is in fluid communication with the elements 11, 121, and 131 along the direction indicated by the arrow of FIG. 1B.

A variety of assays can be used to detect the presence, absence, or concentration of unfolded p53 protein in a sample. For example, unfolded p53 protein (preferentially U-p53^{AZ}) can be detected using binding moieties such as antibodies which can be labeled as well, chemicals that react with the unfolded p53 protein, UV based detection of complex formed by the binding of unfolded p53 protein with binding moieties, electrochemical detection, or any combination thereof. Antibodies that detect unfolded p53 protein are described in PCT/EP2015/072094 which is incorporated by reference in its entirety. An exemplary antibody that binds to unfolded p53 comprises heavy chain CDRs 1, 2 and 3 (SEQ ID NOS: 8, 9 and 10, respectively) and light chain CDRs 1, 2 and 3 (SEQ ID NOS: 11, 12 and 13, respectively).

An exemplary antibody that binds to unfolded p53 comprises the heavy chain variable region (SEQ ID NO: 6) and light chain variable region (SEQ ID NO: 7),

An exemplary antibody that binds to unfolded p53 comprises the heavy chain (SEQ ID NO: 4) and light chain (SEQ ID NO: 5)

An exemplary antibody that binds to unfolded p53 is 2D3A8, which comprises the heavy chain (SEQ ID NO: 4) and light chain (SEQ ID NO: 5), heavy chain variable region (SEQ ID NO: 6) and light chain variable region (SEQ ID NO: 7), heavy chain CDRs 1, 2 and 3 (SEQ ID NOS: 8, 9 and 10, respectively) and light chain CDRs 1, 2 and 3 (SEQ ID NOS: 11, 12 and 13, respectively).

In particular, non-competitive assays can utilize binding moieties that immobilize U-p53, which then allow for the unfolded p53 (for example, U-p53^{AZ}) protein to be detected using additional detectable binding moieties to the unfolded p53 protein.

Alternatively, competitive assays can utilize unfolded p53 protein mimics such as peptide fragments that compete with the unfolded p53 protein for binding to immobilized binding moieties that exhibit specific binding to both the unfolded p53 protein and the unfolded p53 protein mimic. Detection can be either by labeling of the unfolded p53 protein mimic or the use of detectable binding moieties that bind only to the unfolded p53 protein or the unfolded p53 protein mimic. Typically, in noncompetitive formats, a signal is produced if the sample contains the unfolded p53 protein, and no signal is produced if the unfolded p53 protein is not present. In competitive formats, a signal can be produced if no unfolded p53 protein is present and no signal if unfolded p53 protein is present.

“Nitrosilation” as used herein refers to a chemical reaction that adds a nitrosyl group (NO) to an amino acid. For example, S-nitrosylation is a post-translational modification of proteins by which thiol groups (SH groups) of cysteine residues are modified by nitric oxide to generate S-nitrosothiols (protein S-nitrosocysteine), impacting protein function stability, and localization.

“Detectable label”, as used herein, is any substance which is attached to a specific binding member and which is capable of producing a signal that is detectable by visual or instrumental means. Various suitable labels for use in the present invention can include chromogens, catalysts, fluorescent compounds, chemiluminescent compounds, radioactive labels, direct visual labels including colloidal metallic and non-metallic particles, dye particles, enzymes or substrates, or organic polymer latex particles, liposomes or other vesicles containing signal producing substances, and the like. In an alternative signal producing system, the label can be a fluorescent compound as a detectable signal. Fluorescent molecules such as fluorescein, phycobiliprotein, rhodamine and their derivatives and analogs are suitable for use as labels in this reaction.

As used herein, the term “constant magnetic field” refers to the continuous presence of magnetic field which is achieved by the presence of a magnet.

As used herein, the term “dynamic magnetic field” refers to fluctuating magnetic field that can be turned on or off by the user by controlling the amount of electricity that runs through an electromagnet.

As used herein, the term “electromagnet” refers to a type of magnet in which the magnetic field is produced by an electric current. Electromagnets usually consist of wire wound into a coil. A current through the wire creates a magnetic field which is concentrated in the hole, denoting the center of the coil. The magnetic field disappears when the current is turned off.

As used herein, the terms “unfolded” or “conformational variant of” or “misfolded” or “unfolded/misfolded” are used interchangeably. A protein is considered to be misfolded if it cannot achieve its native state of folding. Misfolding can be due to mutations in the amino acid sequence or a disruption of the normal folding process by external factors. A protein is considered to be unfolded (misfolded) when its native conformation is changed due to exposure to one or more of chemical, thermal or mechanical denaturation processes. Unfolded p53 and misfolded p53, are equivalent terms as used herein and refer to an unfolded isoform of the multifunctional protein p53 in plasma which has been known to be present in higher amounts in Alzheimer's Disease patients in comparison with healthy subjects. U-p53^{AZ} is an example of the unfolded/misfolded isoform of p53 protein which is bound by anti-p53 antibody such as 2D3A8. In some instances U-p53AZ is also referred interchangeably as U-p53^{2D3A8+}. See Abate, G., Frisoni, G. B., Bourdon, J C. et al. The pleiotropic role of p53 in functional/dysfunctional neurons: focus on pathogenesis and diagnosis of Alzheimer's disease. Alz Res Therapy 12, 160 (2020) and Unfolded p53: A Potential Biomarker for Alzheimer's Disease, Lanni et al., Journal of Alzheimer's Disease, vol. 12, no. 1, pp. 93-99, 2007.

Cognitive normal subjects have less than 500 pg/ml of unfolded p53 protein (U-p53^{AZ}) which is considered as normal or acceptable levels of U-p53AZ. Patients having different stages of neurogenerative diseases such as dementia or Alzheimer's disease have greater than 500 pgs of U-p53^{AZ}

A “signal producing component”, as used herein, refers to any substance capable of reacting with another assay reagent or unfolded p53 protein (U-p53^{AZ}) to produce a reaction product or signal that indicates the presence of unfolded p53 protein and that is detectable by visual or instrumental means. For example, one or more signal producing components can be used to react with a label and generate the detectable signal, i.e., when the label is an enzyme, amplification of the detectable signal is obtained by reacting the enzyme with one or more substrates or additional enzymes to produce a detectable reaction product.

An “ancillary specific binding member”, as used herein, refers to any member of a specific binding pair which is used in the assay in addition to the specific binding members of the capture reagent and the indicator reagent and which becomes a part of the final binding complex. One or more ancillary specific binding members can be used in an assay. For example, an ancillary specific binding member can be capable of binding unfolded p53 protein, as well as a second specific binding member to which unfolded p53 protein itself could not attach.

“Dementia” as used herein refers a neurological disease state wherein subjects experience symptoms of impairment in memory, communication, and thinking. Possible symptoms of dementia include one or more of cognitive changes such as recent memory loss, difficulty completing familiar and/or routine tasks, problems communicating thoughts to others, disorientation, problems with abstract thinking, misplacing things, and loss of initiative. It can be also accompanied with one or more of psychological changes such as mood changes, personality changes, depression, anxiety, inappropriate behavior, paranoia, agitation and hallucinations, as well as one or more indicators of dementia.

“Indicators of dementia” as used herein refers to the following symptoms which are commonly present in patients with dementia. They include memory loss, hallucination, paranoia, aggressive behavior and depression, as well as difficulty communicating, difficulty with visual and spatial abilities, difficulty with reasoning problem-solving, difficulty handling complex tasks, difficulty with planning and organizing, difficulty with coordination and motor functions, and agitation, confusion and disorientation. In some patients, two or more indicators of dementia are present. In some patients two indicators of dementia are present. In some patients, three indicators of dementia are present. In some patients, four, five, six, seven, eight, nine or ten or more indicators of dementia are present.

“Dementia stages” as used herein refers to developmental stages of the disease which becomes progressively debilitating to the subject. Dementia is roughly split into four stages, (a) Mild cognitive impairment (b) Mild dementia, (c) Moderate dementia and (d) Severe dementia.

“Mild Cognitive Impairment (MCI)” as used herein is a very early stage of dementia characterized by general forgetfulness. This affects many people as they age but it only progresses to dementia for some.

“Mild dementia” as used herein refers to a stage of dementia wherein subject with mild dementia will experience cognitive impairments that occasionally impact their daily life. Symptoms include memory loss, confusion, personality changes, getting lost, and difficulty in planning and carrying out tasks.

“Moderate dementia” as used herein refers to a stage of dementia wherein subject's daily life becomes more challenging, and the individual may need more help. Symptoms are similar to mild dementia but increased. Individuals may need help getting dressed and combing their hair. They may also show significant changes in personality; for instance, becoming suspicious or agitated for no reason. There are also likely to be sleep disturbances.

“Severe dementia” as used herein refers to an advanced stage of dementia wherein the subject's symptoms have worsened considerably. There may be a loss of ability to communicate, and the individual might need full-time care. Simple tasks, such as sitting and holding one's head up become impossible. Bladder control may be lost.

“Progressive dementias” as used herein refers to dementias that progress and are not reversible in nature, but the progression can be slowed down but not prevented. Common examples of progressive dementias include Alzheimer's disease, Vascular dementia, Lewy body dementia, Frontotemporal dementia and Mixed dementia.

“Alzheimer's disease” as used herein refers to neurological disorder which is believed to involve at its early stages progressive dementia. Alzheimer's disease subject has plaques and tangles in their brains. Plaques are clumps of a protein called beta-amyloid, and tangles are fibrous tangles made up of tau protein. It is thought that these clumps damage healthy neurons and the fibers connecting them.

“Vascular dementia” as used herein refers to the most common type of dementia is caused by damage to the vessels that supply blood to your brain. Blood vessel problems can cause strokes or damage the brain in other ways, such as by damaging the fibers in the white matter of the brain. The most common symptoms of vascular dementia include difficulties with problem-solving, slowed thinking, focus and organization. These tend to be more noticeable than memory loss.

“Lewy body dementia” as used herein refers to dementia found in subjects with Lewy bodies. Lewy bodies are abnormal balloon like clumps of protein that have been found in the brain of a subject with Lewy body dementia, Alzheimer's disease and Parkinson's disease. Common symptoms include acting out one's dreams in sleep, visual hallucinations, and problems with focus and attention. Other symptoms include uncoordinated or slow movement, tremors, and rigidity (parkinsonism).

“Frontotemporal dementia” as used herein refers to a group of diseases characterized by the breakdown (degeneration) of nerve cells and their connections in the frontal and temporal lobes of the brain, the areas generally associated with personality, behavior and language. Common symptoms affect behavior, personality, thinking, judgment, and language and movement.

“Mixed dementia” as used herein refers to dementia that is caused by multiple neurological disorders such as Alzheimer's disease, vascular dementia and Lewy body dementia. Elderly subjects over the age of eighty are prone to get mixed dementia.

“Dementia linked disorders” as used herein refers to neurological disorders where subjects having one or more of Huntington's disease, Traumatic brain injury (TBI). Creutzfeldt-Jakob disease and Parkinson's disease exhibit symptoms of dementia.

“Prognosis” as used herein, refers to forecasting of the probable course and outcome of a disease, especially of the chances of recovery. It indicates the prospect of recovery as anticipated from the course of disease.

“Predicting the onset of dementia” as used herein refers to predicting the expected development of dementia, including whether the symptoms of dementia will improve or worsen or remain stable over time. Such a prediction carries with it the expectations of quality of life, such as the ability to carry out daily activities; the potential for complications and associated health issues; and the likelihood of survival (including life expectancy). Typically, a subject seeking prognosis for the onset of dementia have not yet exhibited any of the common symptoms of dementia. Subject seeking prognosis may have one or more close relatives who suffered or suffer from dementia, and therefore might be hereditarily prone to dementia.

“Diagnosis” as used herein, refers to the process of determining by examination the nature and circumstances of a diseased condition.

“Detecting or confirming the presence of dementia” as used herein refers to analyzing a liquid biological sample of a subject having one or more symptoms of dementia for the presence of an analyte such as unfolded p53 protein.

“Onset” refers to beginning or commencement.

“Onset of dementia” in a subject refers to when a subject first exhibits and/or experiences one or more symptoms of dementia.

“Predict” refers to the ability to foretell on the basis of observation, experience, or scientific reason the occurrence of an event.

“Predicting the onset of dementia” refers to determining that a subject having no symptoms of dementia will develop dementia in a follow up period up 10 years by analyzing a patient blood sample for the presence of unfoldedp53 protein (preferentially U-p53^{AZ}).

“Management of dementia” as used herein refers to one or more actions that the subject diagnosed with dementia or predicted to get dementia in the next 10 years can take to slowdown the progression and/or reduce the severity of dementia. These actions include but not limited to (a) Mentally stimulating activities, such as reading, solving puzzles and playing word games, (b) Being physically and socially active, (c) Avoid smoking, (d) Treat high blood pressure, high cholesterol, diabetes, and lower high body mass index (BMI), (e) Increase quality of sleep and (f) Maintain healthy diet and reduce vitamin D deficiency.

“Reversible dementia” as used herein refers to dementia or dementia-like symptoms can be reversed with treatment. Reversible dementia occurs due to one or more of infections and immune disorders, Metabolic problems and endocrine abnormalities, Nutritional deficiencies, Medication side effects, Subdural hematomas, Brain tumors, and Anoxia. Addressing these causes would enable the eradication of dementia symptoms.

“Risk factors for dementia” as used herein refers to one or more factors that are believe to be associated with increasing the chances of getting dementia, these include heavy alcohol use, elevated cardiovascular risk due to high body mass index (BMI), depression, diabetes, smoking, sleep apnea and vitamin deficiencies involving low levels of vitamin D, vitamin B-6, vitamin B-12 and folate.

“Symptoms of dementia” as used herein refers to one or more cognitive and/or psychological changes that signal the presence or onset of dementia. Examples of cognitive change include one or more of memory loss, difficulty communicating, difficulty with visual and spatial abilities, difficulty with reasoning problem-solving, difficulty handling complex tasks, difficulty with planning and organizing, difficulty with coordination and motor functions, and agitation, confusion and disorientation. Examples of psychological change include one or more of depression, anxiety, paranoia, and hallucinations.

“Genetic predisposition to dementia” as used herein refers to a condition wherein the subject has close family relatives such as siblings, parents or grandparents who have been diagnosed with dementia. It also includes subject who has abnormal expression of or have mutations that affect function of one or more genes that are believed to play a role in the onset of dementia. These dementia related genes are selected from the group consisting of APOE4, ABCA7, CLU, CR1, PICALM, PLD3, TREM2, SORL1, APP, PSEN1 and PSEN2.

“Cognitive Decline” as used herein refers to a condition wherein the individual shows a reduced ability to learn new information, an increase in becoming distracted, and a slower mental process, and memory loss.

“Subjective memory complaints” as used herein refers to a condition wherein an individual experiences self-reported problems with memory that may or may not present with objective cognitive impairment (measured via tests and assessments).

“Flow path” as used herein refers to the direction in which the sample containing U-p53^{AZ} flows across the test strip. The sample typically travels across the strip through capillary action from the sample zone to the detection zone.

“Sample zone” as used herein refers to the region in the device wherein the sample from the subject in introduced into the device. Optionally, the sample zone can contain lysis buffers, DNAses, proteases, lipases, desiccants, or other reagents that can prepare a sample for analysis. For example, the sample zone can contain a protease that can reduce the viscosity of sample such as saliva or chemicals such as acetonitrile or methanol to enable separation of plasma from blood.

“Reagent zone” as used herein refers to the region in the device wherein the antibody that binds to U-p53 is contained therein.

“Test zone”, “detection zone”, “capture zone” are equivalent terms used herein to refer to the region of the test strip of the device in which the capture agent that binds to unfolded p53 (preferentially U-p53^{AZ}) and-anti-U-p53 antibody complex is contained. The test zone optionally comprises a control line and a result line.

“Loading port” as used herein refers to region within the sample zone through which sample is applied to the device. The sample introduced through the loading port can optionally get processed in the sample zone by action of proteases or chemicals such as acetonitrile.

“Device housing” as used herein refers to the support framework within which the test strip can be positioned. The housing that contains the test strip can optionally have a viewing window to observe the results of the assay. The test strip can be aligned within the housing such that test lines and/or control lines on the test strip are positioned beneath the viewing window. The viewing window can have an optically transparent material, or the viewing window can be a cut out in the test device housing.

“Test line” or “Test band” as used herein refers to bright colored line or gold or silver band that appears when the sample tested contains the analyte of interest (unfolded p53) indicating a positive result. The absence of a test band indicates a negative result. The test band can also be used to determine the presence, absence, or concentration of unfolded p53in the sample

“Control line” or “Control band” as used herein refers to bright colored line or gold or silver band that appears to indicate that the reagents used in the device are functional. The control lines can comprise a positive control line and a negative control line. Reagents that can bind to the control line can be disposed in the sample zone or the reagent zone in a dry state.

### Devices and Methods

The devices and methods according to the invention can be used to determine the presence, absence, or concentration of unfolded p53in a biological sample. A sample can be collected from a test subject, such as a human or an animal, and then analyzed using the devices and methods described herein. A sample can be introduced directly onto the test strip, as described herein, at a sample loading zone. Alternatively, a sample can be incubated with a first set of reagents to form a mixture, and the mixture can then be applied to the sample loading zone. The test strip has two additional zones, a reagent zone and a test zone. The three zones are in fluid communication such that introduction of the liquid sample to the sample zone commences flow along the test strip to the reagent zone and then to the detection zone.

When the sample is directly applied to the device in the sample loading zone, the sample loading zone optionally may contain chemical agents which permit chemical processing (digestion, precipitation and removal) of the unwanted components of the biological sample. This optional processing step can include the deposit of chemicals such as acetonitrile, methanol, or toluene which may be embedded in the nitrocellulose membrane (which is a porous medium present in the device). In some embodiments, the chemicals such as acetonitrile are in a reservoir which can be released by finger pressure or pin prick which then exposes the chemical to the sample. In an example, the use of acetonitrile in the sample loading zone permits precipitation or digestion of unwanted components, such as albumins, platelets, red blood cells, of a blood sample; the digested or precipitated components will not be carried along the flow path of the test strip to the detection zone. Therefore, such chemical digestion permits better diffusion along the test strip.

The sample can move through capillary action or by diffusion from the sample zone to a first reagent zone that has a one or more reagents. The sample comprising unfolded p53as the analyte is introduced into the sample zone and flows to the first reagent zone where it encounters one or more reagents, for example, a mobile binding agent specific for unfolded p53, where the mobile binding agent is labeled with a label that is detectable, thereby forming a binding complex, as described herein.

Once the complex with the detectable label is formed, the complex can flow along the flow path to a test (detection) zone. The test zone can have one or more tests lines and the control zone can have one or more control lines that can be used to determine the presence, absence, or concentration of unfolded p53 (for example U-p53^{AZ}) in the sample. The results of the assay can be qualitative or quantitative. For example, the presence of a line on a test zone can indicate either the presence or absence of unfolded p53 (for example U-p53^{AZ}) in the sample. Alternatively, the concentration of Unfolded p53 (for example U-p53^{AZ}) in the sample can be determined by analyzing the test line. For example, a greater amount of detectable label can be present on the test line when greater amounts of Unfolded p53 are contained within the sample. The test line can be analyzed visually or spectrophotometrically using methods known to those skilled in the art to quantitatively determine the concentration of the unfolded p53 in the sample.

The methods of the invention can provide for increased assay sensitivity and accuracy. The increased sensitivity and accuracy can be a result of allowing the sample to incubate with the reagents in the second reagent zone for a sufficient period of time to react with Unfolded p53 in the sample. In some embodiments, the concentration of an analyte in the sample ranges from 10-10000 pg/ml.

In some embodiments the concentration of the Unfolded p53 binding agent such as an antibody on the device ranges from 1-30 ugs.

In some embodiments, the method comprises adding subject liquid sample to the sample entry region or sample zone of the strip. Analyte (Unfolded p53, preferably U-p53^{AZ}) contained in the liquid sample migrates along the test strip in the fluid flow direction until the it enters the testing region. If unfolded p53 (U-p53^{AZ}) is present in the sample, it will bind to the mobile unfolded p53 binding agent such as anti-Up53 antibody in the reagent zone and migrate along the flow path to the test zone where it becomes immobilized, and a sufficient amount of the labeled complex containing the Unfolded p53 (for example U-p53^{AZ}) accumulates to form a visual signal indicating the present of at least 600±100 pg/ml of unfolded p53 in the biological sample.

The label selected for use in the device will generate a visually detectable signal. The signal does not need to be a colorimetric signal but can, for example, generate another type of signal, such as an electronic image-like positive sign or an emoji face in the testing zone. Alternatively, the label may form a minus sign upon interaction with a control zone to indicate a negative test result, i.e. no Unfolded p-53 (U-p53^{AZ}) present in the sample. If Unfolded p-53 (U-p53^{AZ}) is present in the detection zone, the label may be generated in a format wherein the signal is perpendicular to a control line, thereby completing formation of a plus sign + to indicate a positive test result, i.e. Unfolded-p53is present in the test sample.

In some aspects, the disclosure provides a data reporting module in combination with the lateral flow device. In some embodiments, the data reporting module comprises a clip-on device that links to the camera on a smartphone belonging to the user accessing the lateral flow test device and would clip in the lateral flow test device. In other embodiments, the clip-on device only connects to the camera of the smartphone to scan the signal generated by the lateral flow device. The clip-on device then accesses the internet to transmit the signal data to an application that can generate a risk score for the user based on his or her test result. In some embodiments, the application that generates the risk score is healthy.io (details of which are found in the website of the application titled “healthy.io/eu”.). The generation of risk score initiates the process of creation of official report in the application which is then sent to the physician or medical care provider of the user to enable them to update the user's medical records to reflect the results of the test performed through the lateral flow device.

In some embodiments, the application would provide a quality score to the test result based on the density of the control line on the lateral flow device or the movement of the colored bound complex (U-p53AZ-anti-p-53 antibody) across the boundary. The risk score can range from 1 to 10, with 1 being lowest risk for decline into dementia and 10 being the highest risk for developing dementia. The application can also evaluate the signal data to determine the quality of the signal, if the application deems the signal to be insufficient, it can optionally contact the user to retake the test or inform the medical provider monitoring the user to repeat the test. In some embodiments, the risk score of 5 indicates that the user is in a position to prevent decline into dementia by engaging lifestyle changes such as reducing inflammation, undertaking exercises and losing weight etc. It is contemplated that the user after following lifestyle changes would repeat the test on the lateral flow device to ensure that he or she is receiving lower risk score than the risk score achieved before lifestyle change.

In some embodiments, risk score of decline based on the amount of u-p53 detected with higher amounts of U-p53 in the sample corelate with higher risk for developing dementia. It is expected that higher amounts of U-p53^{AZ} in the sample will generate stronger signal in the lateral flow device. In some embodiments, the application doesn't transmit the risk score to the patient but only sends a report to the medical practitioner.

In some embodiments, the data from the scanned lateral flow device can be uploaded through the application to a cloud storage database for global performance and quality control. This would allow a long-term analysis of the user's progress or decline over time. In some embodiments, the cloud storage system would facilitate the creation of a database of the patient test data for trend lines on medical benefits around the diseases, other dementia, treatment, performance, and demographics for a large group of users.

A test device described herein can be used for detecting one or more analytes in a sample. While preferable embodiments of the invention have been shown and described herein, it will be obvious to those skilled in the art that such embodiments are provided by way of example only. The devices and/or methods of the invention can be employed individually or combined with other devices, methods, and/or systems in manners known to those skilled in the arts for detection of one or more analytes in a sample.

The binding agent can be labeled using one or more detectable labels. Commonly used detectable labels include but are not limited to gold nano particles, colloidal gold particles, colloidal platinum particles, colloidal silver particles, colored latex particles, carbon nanotubes, fluorescent labels, quantum dots, ruthenium complexes, paramagnetic labels, enzyme labels and latex nanocomposite particles.

Details of latex nanocomposite labels and methods of use are found in “Metal (Au, Pt) Nanoparticle—Latex Nanocomposites as Probes for Immunochromatographic Test Strips with Enhanced Sensitivity”, Matsumura et al., ACS Appl. Mater. Interfaces 2018, 10, 31977-31987. Details on commonly used fluorescent labels can be found in “Fluorescence-Based Bioassays for the Detection and Evaluation of Food Materials”, Nishi et al. Sensors 2015, 15, 25831-25867.

Details of lateral flow devices which use gold nano particles, colloidal gold particles, colloidal platinum particles, colloidal silver particles, and colored latex particles are found in Lateral flow assays, Koczula et al., Essays in Biochemistry (2016) 60 111-120; Shyu, R. H., Shyu, H. F., Liu, H. W. and Tang, S. S. (2002) Colloidal gold-based immunochromatographic assay for detection of ricin. Toxicon 40, 255-258; Dai, M.; Gong, Y.; Liu, A.; Zhang, L.; Lin, J.; Zhang, M.; Yu, X. Development of a Colloidal Gold-Based Lateral-Flow Immunoassay for the Rapid Detection of Phenylethanolamine A in Swine Urine. Anal. Methods 2015, 7, 4130-4137; Wu, W.-D.; Li, M.; Chen, M.; Li, L.-P.; Wang, R.; Chen, H.-L.; Chen, F.-Y.; Mi, Q.; Liang, W.-W.; Chen, H.-Z. Development of a Colloidal Gold Immunochromatographic Strip for Rapid Detection of Streptococcus agalactiae in Tilapia. Biosens. Bioelectron. 2017, 91, 66-69; Mao, X.; Wang, W.; Du, T. E. Dry-Reagent Nucleic Acid Biosensor Based on Blue Dye Doped Latex Beads and Lateral Flow Strip. Talanta 2013, 114, 248-253.

Details of lateral flow devices which use fluorescent labels, quantum dots, ruthenium complexes, paramagnetic labels, and enzyme labels are found in Nishi et al., Sensors 2015, 15, 25831-25867; Liu, T.; Liu, B.; Zhang, H.; Wang, Y. The fluorescence bioassay platforms on quantum dots nanoparticles. J. Fluoresc. 2005, 15, 729-733; Medintz, I. L.; Uyeda, H. T.; Goldman, E. R.; Mattoussi, H. Quantum dot bioconjugates for imaging, labelling and sensing. Nat. Mater. 2005, 4, 435-446; Zhang, Y.; Wang, T. H. Quantum dot enabled molecular sensing and diagnostics. Theranostics, 2012, 2, 631-654; Liu, C., Jia, Q., Yang, C., Qiao, R., Jing, L., Wang, L. et al. (2011) Lateral flow immunochromatographic assay for sensitive pesticide detection by using Fe3O4 nanoparticle aggregates as color reagents. Anal. Chem. 83, 6778-678; Lin, Y. Y., Wang, J., Liu, G., Wu, H., Wai, C. M. and Lin, Y. (2008) A nanoparticle label/immunochromatographic electrochemical biosensor for rapid and sensitive detection of prostate-specific antigen. Biosens. Bioelectron. 23, 1659-1665; Song, L. W., Wang, Y. B., Fang, L. L., Wu, Y., Yang, L., Chen, J. Y. et al. (2015) Rapid fluorescent lateral-flow immunoassay for hepatitis B virus genotyping, Anal. Chem. 87, 5173-5180; Venkatraman, V. and Steckl, A. J. (2015) Integrated OLED as excitation light source in fluorescent lateral flow immunoassays. Biosens. Bioelectron. 74, 150-155; Wang, D. B., Tian, B., Zhang, Z. P., Wang, X. Y., Fleming, J., Bi, L. J. et al. (2015) Detection of Bacillus anthracis spores by super-paramagnetic lateral-flow immunoassays based on “Road Closure”. Biosens. Bioelectron. 67, 608-614; Mirasoli, M., Buragina, A., Dolci, L. S., Guardigli, M., Simoni, P., Montoya, A. et al. (2012) Development of a chemiluminescence-based quantitative lateral flow immunoassay for on-field detection of 2,4,6-trinitrotoluene. Anal. Chim. Acta 721, 167-17; Maiolini, E., Ferri, E., Pitasi, A. L., Montoya, A., Di Giovanni, M., Errani, E. et al. (2014) Bisphenol A determination in baby bottles by chemiluminescence enzyme-linked immunosorbent assay, lateral flow immunoassay and liquid chromatography tandem mass spectrometry. Analyst 139, 318-324.

The p53 protein sequence (SEQ ID NO: 3) is depicted in FIG. 8, where the DNA binding domain (DBD) is highlighted in grey, the linear epitope (SEQ ID NO: 1) recognized by the 2D3A8 antibody is underlined.

SEQUENCES

```
SEQ ID NO: 1-Linear Epitope
Arg Arg Thr Glu Glu Glu Asn Leu Arg Lys Lys Gly Glu Pro His His
1 5 10 15
SEQ ID NO: 2-Immunization Peptide
Cys Arg Thr Glu Glu Glu Asn Leu Arg Lys Lys Gly Glu Pro His His
1 5 10 15
SEQ ID NO: 3-p53 Protein
Met Glu Glu Pro Gln Ser Asp Pro Ser Val Glu Pro Pro Leu Ser Gln
1 5 10 15
Glu Thr Phe Ser Asp Leu Trp Lys Leu Leu Pro Glu Asn Asn Val Leu
                        20 25 30
Ser Pro Leu Pro Ser Gln Ala Met Asp Asp Leu Met Leu Ser Pro Asp
                35 40 45
Asp Ile Glu Gln Trp Phe Thr Glu Asp Pro Gly Pro Asp Glu Ala Pro
        50 55 60
Arg Met Pro Glu Ala Ala Pro Pro Val Ala Pro Ala Pro Ala Ala Pro
65 70 75 80
Thr Pro Ala Ala Pro Ala Pro Ala Pro Ser Trp Pro Leu Ser Ser Ser
                                85 90 95
Val Pro Ser Gln Lys Thr Tyr Gln Gly Ser Tyr Gly Phe Arg Leu Gly
                        100 105 110
Phe Leu His Ser Gly Thr Ala Lys Ser Val Thr Cys Thr Tyr Ser Pro
                115 120 125
Ala Leu Asn Lys Met Phe Cys Gln Leu Ala Lys Thr Cys Pro Val Gln
        130 135 140
Leu Trp Val Asp Ser Thr Pro Pro Pro Gly Thr Arg Val Arg Ala Ala
145 150 155 160
Ile Tyr Lys Gln Ser Gln His Met Thr Glu Val Val Arg Arg Cys Pro
                                165 170 175
His His Glu Arg Cys Ser Asp Ser Asp Gly Leu Ala Pro Pro Gln His
                        180 185 190
Leu Ile Arg Val Glu Gly Asn Leu Arg Val Glu Tyr Leu Asp Asp Arg
                195 200 205
Asn Thr Phe Arg His Ser Val Val Val Pro Tyr Glu Pro Pro Glu Val
        210 215 220
Gly Ser Asp Cys Thr Thr Ile His Tyr Asn Tyr Met Cys Asn Ser Ser
225 230 235 240
Cys Met Gly Gly Met Asn Arg Arg Pro Ile Leu Thr Ile Ile Thr Leu
                                245 250 255
Glu Asp Ser Ser Gly Asn Leu Leu Gly Arg Asn Ser Phe Glu Val Arg
                        260 265 270
Val Cys Ala Cys Pro Gly Arg Asp Arg Arg Thr Glu Glu Glu Asn Leu
                275 280 285
Arg Lys Lys Gly Glu Pro His His Glu Leu Pro Pro Gly Ser Thr Lys
        290 295 300
Arg Ala Leu Pro Asn Asn Thr Ser Ser Ser Pro Gln Pro Lys Lys Lys
305 310 315 320
Pro Leu Asp Gly Glu Tyr Phe Thr Leu Gln Ile Arg Gly Arg Glu Arg
                                325 330 335
Phe Glu Met Phe Arg Glu Leu Asn Glu Ala Leu Glu Leu Lys Asp Ala
                        340 345 350
Gln Ala Gly Lys Glu Pro Gly Gly Ser Arg Ala His Ser Ser His Leu
                355 360 365
Lys Ser Lys Lys Gly Gln Ser Thr Ser Arg His Lys Lys Leu Met Phe
        370 375 380
Lys Thr Glu Gly Pro Asp Ser Asp
385 390
SEQ ID NO: 4-Heavy chain of 2D3A8 antibody
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                        20 25 30
Val Met His Trp Val Lys Gln Lys Pro Gly Gln Gly Leu Glu Trp Ile
                35 40 45
Gly Tyr Ile Asn Pro Tyr Asn Asp Gly Thr Lys Tyr Asn Glu Lys Phe
        50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                                85 90 95
Ala Arg Gly Gly Tyr Tyr Ala Met Asp Tyr Trp Gly Gln Gly Thr Ser
                        100 105 110
Val Thr Val Ser Ser Glu Ser Gln Ser Phe Pro Asn Val Phe Pro Leu
                115 120 125
Val Ser Cys Glu Ser Pro Leu Ser Asp Lys Asn Leu Val Ala Met Gly
        130 135 140
Cys Leu Ala Arg Asp Phe Leu Pro Ser Thr Ile Ser Phe Thr Trp Asn
145 150 155 160
Tyr Gln Asn Asn Thr Glu Val Ile Gln Gly Ile Arg Thr Phe Pro Thr
                                165 170 175
Leu Arg Thr Gly Gly Lys Tyr Leu Ala Thr Ser Gln Val Leu Leu Ser
                        180 185 190
Pro Lys Ser Ile Leu Glu Gly Ser Asp Glu Tyr Leu Val Cys Lys Ile
                195 200 205
His Tyr Gly Gly Lys Asn Arg Asp Leu His Val Pro Ile Pro Ala Val
        210 215 220
Ala Glu Met Asn Pro Asn Val Asn Val Phe Val Pro Pro Arg Asp Gly
225 230 235 240
Phe Ser Gly Pro Ala Pro Arg Lys Ser Lys Leu Ile Cys Glu Ala Thr
                                245 250 255
Asn Phe Thr Pro Lys Pro Ile Thr Val Ser Trp Leu Lys Asp Gly Lys
                        260 265 270
Leu Val Glu Ser Gly Phe Thr Thr Asp Pro Val Thr Ile Glu Asn Lys
                275 280 285
Gly Ser Thr Pro Gln Thr Tyr Lys Val Ile Ser Thr Leu Thr Ile Ser
        290 295 300
Glu Ile Asp Trp Leu Asn Leu Asn Val Tyr Thr Cys Arg Val Asp His
305 310 315 320
Arg Gly Leu Thr Phe Leu Lys Asn Val Ser Ser Thr Cys Ala Ala Ser
                                325 330 335
Pro Ser Thr Asp Ile Leu Thr Phe Thr Ile Pro Pro Ser Phe Ala Asp
                        340 345 350
Ile Phe Leu Ser Lys Ser Ala Asn Leu Thr Cys Leu Val Ser Asn Leu
                355 360 365
Ala Thr Tyr Glu Thr Leu Asn Ile Ser Trp Ala Ser Gln Ser Gly Glu
        370 375 380
Pro Leu Glu Thr Lys Ile Lys Ile Met Glu Ser His Pro Asn Gly Thr
385 390 395 400
Phe Ser Ala Lys Gly Val Ala Ser Val Cys Val Glu Asp Trp Asn Asn
                                405 410 415
Arg Lys Glu Phe Val Cys Thr Val Thr His Arg Asp Leu Pro Ser Pro
                        420 425 430
Gln Lys Lys Phe Ile Ser Lys Pro Asn Glu Val His Lys His Pro Pro
                435 440 445
Ala Val Tyr Leu Leu Pro Pro Ala Arg Glu Gln Leu Asn Leu Arg Glu
        450 455 460
Ser Ala Thr Val Thr Cys Leu Val Lys Gly Phe Ser Pro Ala Asp Ile
465 470 475 480
Ser Val Gln Trp Leu Gln Arg Gly Gln Leu Leu Pro Gln Glu Lys Tyr
                                485 490 495
Val Thr Ser Ala Pro Met Pro Glu Pro Gly Ala Pro Gly Phe Tyr Phe
                        500 505 510
Thr His Ser Ile Leu Thr Val Thr Glu Glu Glu Trp Asn Ser Gly Glu
                515 520 525
Thr Tyr Thr Cys Val Val Gly His Glu Ala Leu Pro His Leu Val Thr
        530 535 540
Glu Arg Thr Val Asp Lys Ser Thr Gly Lys Pro Thr Leu Tyr Asn Val
545 550 555 560
Ser Leu Ile Met Ser Asp Thr Gly Gly Thr Cys Tyr
                                565 570
SEQ ID NO: 5-Light chain of 2D3A8 antibody
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
                        20 25 30
Leu Asn Trp Tyr Gln Gln Lys Pro Asp Gly Thr Val Lys Leu Leu Ile
                35 40 45
Tyr Tyr Thr Ser Arg Leu His Ser Gly Val Pro Ser Arg Phe Ser Gly
        50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                                85 90 95
Thr Phe Gly Gly Gly Thr Lys Leu Glu Ile Lys Arg Ala Asp Ala Ala
                        100 105 110
Pro Thr Val Ser Ile Phe Pro Pro Ser Ser Glu Gln Leu Thr Ser Gly
                115 120 125
Gly Ala Ser Val Val Cys Phe Leu Asn Asn Phe Tyr Pro Lys Asp Ile
        130 135 140
Asn Val Lys Trp Lys Ile Asp Gly Ser Glu Arg Gln Asn Gly Val Leu
145 150 155 160
Asn Ser Trp Thr Asp Gln Asp Ser Lys Asp Ser Thr Tyr Ser Met Set
                                165 170 175
Ser Thr Leu Thr Leu Thr Lys Asp Glu Tyr Glu Arg His Asn Ser Tyr
                        180 185 190
Thr Cys Glu Ala Thr His Lys Thr Ser Thr Ser Pro Ile Val Lys Ser
                195 200 205
Phe Asn Arg Asn Glu Cys
        210
SEQ ID NO: 6-Heavy chain variable region of 2D3A8 antibody
Glu Val Gln Leu Gln Gln Ser Gly Pro Glu Leu Val Lys Pro Gly Ala
1 5 10 15
Ser Val Lys Met Ser Cys Lys Ala Ser Gly Tyr Thr Phe Thr Ser Tyr
                        20 25 30
Val Met His Trp Val Lys Gln Lys Pro Gly Gln Gly Leu Glu Trp Ile
                35 40 45
Gly Tyr Ile Asn Pro Tyr Asn Asp Gly Thr Lys Tyr Asn Glu Lys Phe
        50 55 60
Lys Gly Lys Ala Thr Leu Thr Ser Asp Lys Ser Ser Ser Thr Ala Tyr
65 70 75 80
Met Glu Leu Ser Ser Leu Thr Ser Glu Asp Ser Ala Val Tyr Tyr Cys
                                85 90 95
Ala Arg Gly Gly Tyr Tyr Ala Met Asp Tyr Trp Gly Gln Gly Thr Ser
                        100 105 110
Val Thr Val Ser Ser
                115
SEQ ID NO: 7-Light chain variable region of 2D3A8 antibody
Asp Ile Gln Met Thr Gln Thr Thr Ser Ser Leu Ser Ala Ser Leu Gly
1 5 10 15
Asp Arg Val Thr Ile Ser Cys Arg Ala Ser Gln Asp Ile Ser Asn Tyr
                        20 25 30
Leu Asn Trp Tyr Gln Gln Lys Pro Asp Gly Thr Val Lys Leu Leu Ile
                35 40 45
Tyr Tyr Thr Ser Arg Leu His Ser Gly Val Pro Ser Arg Phe Ser Gly
        50 55 60
Ser Gly Ser Gly Thr Asp Tyr Ser Leu Thr Ile Ser Asn Leu Glu Gln
65 70 75 80
Glu Asp Ile Ala Thr Tyr Phe Cys Gln Gln Gly Asn Thr Leu Pro Tyr
                                85 90 95
Thr Phe Gly Gly Gly Thr Lys Leu Glu Ile Lys
                        100 105
SEQ ID NO: 8-Heavy chain CDR1 of 2D3A8 antibody
Ser Tyr Val Met His
1 5
SEQ ID NO: 9-Heavy chain CDR2 of 2D3A8 antibody
Tyr Ile Asn Pro Tyr Asn Asp Gly Thr Lys Tyr Asn Glu Lys Phe Lys
1 5 10 15
Gly
SEQ ID NO: 10-Heavy chain CDR3 of 2D3A8 antibody
Gly Gly Tyr Tyr Ala Met Asp Tyr
1 5
SEQ ID NO: 11-Light chain CDR1 of 2D3A8 antibody
Arg Ala Ser Gln Asp Ile Ser Asn Tyr Leu Asn
1 5 10
SEQ ID NO: 12-Light chain CDR2 of 2D3A8 antibody
Tyr Thr Ser Arg Leu His Ser
1 5
SEQ ID NO: 13-Light chain CDR3 of 2D3A8 antibody
Gln Gln Gly Asn Thr Leu Pro Tyr Thr
1 5
```

### EXAMPLES

### Example 1—Preparation of an Antibody Comprising CDRs Having SEQ ID NOS: 8-13

The process of producing an antibody having a specified set of CDRs is well known in art. For example, Frenzel et al. (Expression of recombinant antibodies, Front. Immunol., 29 Jul. 2013) describes different expression systems that can be used for the production of antibodies having a specific sequence. The antibodies can be produced using recombinant immunoglobulin expression technology. The recombinant production of immunoglobulin molecules, including humanized antibodies are described in U.S. Pat. No. 4,816,397 (Boss et al.), U.S. Pat. Nos. 6,331,415 and 4,816,567 (both to Cabilly et al.), U.K. patent GB 2,188,638 (Winter et al.), and U.K. patent GB 2,209,757. Techniques for the recombinant expression of immunoglobulins, including humanized immunoglobulins, can also be found, in Goeddel et al. “Gene Expression Technology Methods in Enzymology”. Vol. 185 Academic Press, (1991), and Borreback, “Antibody Engineering”. W. H. Freeman (1992). Additional information concerning the generation, design and expression of recombinant antibodies can be found in Mayforth, “Designing Antibodies”. Academic Press, San Diego (1993).

An exemplary process for the production of the recombinant antibodies can include the following: a) constructing, by conventional molecular biology methods, an expression vector that encodes and expresses an antibody heavy chain of the anti-U-p53 antibody comprising heavy chain CDR sequences (1,2,3) denoted by SEQ ID NO: 8, 9 and 10), b) constructing, by conventional molecular biology methods, an expression vector that encodes and expresses an antibody light chain of the anti-U-p53 antibody comprising light chain CDR sequences (1,2,3) denoted by SEQ ID NO: 11, 12 and 13), c) transferring the expression vectors to a host cell by conventional molecular biology methods to produce a transfected host cell for the expression of chimeric antibodies; and d) culturing the transfected cell by conventional cell culture techniques so as to produce chimeric antibodies. Further details can be found in Advances in recombinant antibody manufacturing, Kunert et al., Appl Microbiol Biotechnol. 2016; 100:3451-3461.

### Example 2—Conjugation of Unfolded p53 Binding Antibody to Magnetic Beads

An antibody specific for U-p53, for example the 2D3A8 antibody whose heavy and light chain amino acid sequences are presented herein as SEQ ID NOS: 4 and 5, is conjugated with beads following manufacturer protocols. Several commercial options for conjugating antibodies or proteins to latex beads are described in the art. [Two-Color Lateral Flow Assay for Multiplex Detection of Causative Agents Behind Acute Febrile Illnesses; Lee et al., Anal Chem. 2016 Sep. 6; 88 (17): 8359-8363.]. In addition, commercially available latex bead conjugations protocols include kits available from Pierce-Thermofisher, LATEX conjugation kit—Innova Biosciences Ltd, Sigma, Prospect and GenScript.

The 2D3A8 antibody is conjugated to Protein L magnetic beads following instructions in the Pierce™ Protein L Magnetic Beads (Catalog No: 88849) kit. These kits comprise recombinant Protein L, covalently attached to a blocked magnetic bead surface, that selectively binds mouse and human antibodies through kappa light chains.

50 μL (0.50 mg) of Pierce Protein L Magnetic Beads is added to a 1.5 mL microcentrifuge tube. To which 150 μL of Tris-buffered saline (TBS, Product No. 28360) containing 0.05% Tween-20 Detergent is added and gently vortexed to mix. The magnetic beads are collected using a magnetic stand and the supernatant TBS is removed. The process is repeated a second time to prepare the beads for binding to the antibody (2D3A8). The washed beads are then mixed with 500 μl of antibody and are allowed to at room temperature with mixing for 1 hour. The magnetic beads are collected using a magnetic stand and the supernatant TBS is removed. The washing process is repeated again to remove any remnants of antibody that are not conjugated with beads resulting in a slurry of beads conjugated to 2D3A8 antibody.

### Example 3—Detection of Unfolded p53 (U-p53^{AZ}) Using 2D3A8 Antibody Conjugated to Magnetic Beads

In order to demonstrate that 2D3A8-conjugated are able to detect unfolded p53 protein (U-p53^{AZ})) in blood samples, Unfolded labeled p-53 (Labeled -U-p53^{AZ}) protein was spiked at different concentrations into human blood samples that were initially devoid of Unfolded p-53 (U-p53^{AZ}) protein. These Unfolded p-53 (U-p53^{AZ})-spiked human blood samples were mixed with 2D3A8 conjugated beads at room temperature.

Eight separate blood samples were tested for detection of Unfolded labeled p-53 (labelled U-p53^{AZ}) in the spiked samples. The results of the recovery are shown in FIG. 9. The results indicate that over 96% of Unfolded labeled p53 protein in the plasma samples was bound and thus recovered by 2D3A8 conjugated beads. The quantization of the protein was carried out by its specific labeled peptide “TEEENLR”.

### Example 4—Detection of Unfolded p-53 (U-p53^{AZ}) in Blood Samples Over Time

2D3A8-conjugated beads retain their ability to bind and thereby recover Unfolded p-53 protein in blood samples over a period of time. The stability of 2D3A8 conjugated beads and the ability to capture Unfolded p53 (U-p53^{AZ}) protein in the spiked blood samples was evaluated until 22 days from preparation of the stock reagent. The assays were repeated following the protocols described in Example 3.

The results of the stability assays are shown in FIG. 10 and they indicate that 2D3A8 conjugated beads were stable for over 22 days after the conjugation to the bead occurred. The 2D3A8 conjugated beads remained viable in their ability to bind/capture U-p53^{AZ} protein. They exhibited over 90% recovery rates.

The same protocols are repeated and the stability of 2D3A8 conjugated beads is evaluated at 30 days, 45 days, 60 day, 90 days, 120 days and 180 days post preparation of stock reagent. The results are expected to indicate that 2D3A8 conjugated beads are stable and retain their activity to bind/capture Unfolded p-53 (preferably U-p53^{AZ}) over a period of at least 6 months.

### Example 5—Preparation of Lateral Flow Device for Detection of Unfolded U-p53 (Preferably U-p53^{AZ})

The lateral flow device comprises a dry porous carrier to which a liquid sample, e.g. blood, containing an analyte such as Unfolded p53 (U-p53^{AZ}) can be applied. The device contains a labelled specific binding reagent or an unlabeled specific binding agent which is freely mobile in the porous carrier when in the moist state. The specific binding reagent being capable of participating in either a sandwich reaction or a competition reaction in the presence of Unfolded p-53 (u-p53^{AZ}), in which prior to the application to the device of a liquid sample containing the analyte, the labelled specific binding reagent is retained in the dry state in a macroporous body, e.g., of plastics material having a pore size of 10 microns or greater, through which the applied liquid sample must pass en route to the porous carrier material, the labelled specific binding reagent being freely soluble or dispersible in any liquid sample which enters the macroporous body.

Referring to FIG. 1C, it can be seen that housing 100 is of hollow construction. Porous sample collector 106 extends into housing 100. The inner end 112 of sample collector 106 is recessed to accommodate a macroporous body 113 of plastics material. Aqueous liquid sample applied to collector 106 can pass freely into macroporous body 113, rapidly saturating it. In turn, macroporous body 113 is in liquid permeable contact with a strip of porous carrier material 114. The housing is constructed of an upper half 110 and a lower half 120 and strip 114 overlap to ensure that there is adequate contact between these two components and that a liquid sample applied to sample collector 106 can permeate via macroporous body 113 and into strip 114. Strip 114 extends further into housing 100.

To help ensure that no liquid sample reaches Strip 114 without first passing through macroporous body 113, a gap 115 can be left in the housing 100 by arranging for the strip 114 to overlap macroporous body 113 only partially. Strip 114 is “backed” by a supporting strip 116 formed of transparent moisture-impermeable plastics material. Strip 114 extends beyond apertures 108 and 109. Means are provided within housing 100 by 117 and 118 to hold strip 114 firmly in place. In this respect, the internal constructional details of the housing are not a significant aspect of the invention as long as the strip is held firmly in place within the housing, sample collector 106 is firmly retained in the housing, and adequate fluid permeable contact is maintained between sample collector 106, macroporous body 113 and strip 114. The transparent backing strip 116 lies between strip 114 and apertures 108 and 109 and can act as a seal against ingress of moisture from outside the housing 100 via these apertures. If desired, the residual space 119 within the housing can contain moisture-absorbent material, such as silica gel, to help maintain the strip 114 in the dry state during storage. The reagent-containing detection zone in strip 114 is not depicted in FIG. 2, but the zone containing the immobilized unlabeled reagent will lie in the region exposed through aperture 108 in order that when the device has been used in an assay, the result can be observed through aperture 108. Aperture 109 provides means through which a control zone containing further reagents which may enable the adequate permeation of sample through the strip to be observed.

In operation, the protective cap 103 is removed from the holder and sample collector 106 is exposed to a liquid sample e.g. by a pin prick to draw blood which is deposited onto the sample collector. Alternatively, the sample collector can have a pin embedded which gets exposed when the subject places their finger on to the sample pad and applies pressure. After exposing sample collector 106 to the liquid sample for a time sufficient to ensure that the collector 106 is saturated with the sample, the cap 103 can be replaced and the device placed aside by the user for an appropriate period time (e.g. two or three minutes) while the sample permeates test strip 114 to provide the analytical result. After the appropriate time, the subject can observe the test strip through apertures 108 and 109 and can ascertain whether the assay has been completed by observing the control zone through aperture 109, and can ascertain the result of the assay by observing the second zone through aperture 108.

During manufacture, the device can be readily assembled from, for example, plastics material with the housing 100 being molded in two parts (e.g. upper and lower halves 110 and 120) which can be securely fastened together (e.g. by ultrasonic welding) after the sample collector, macroporous body and test strip have been placed within one of the halves and then sandwiched between the two halves. The act of forming this sandwich construction can be used to “crimp” the sample collector macroporous body and test strip together to ensure adequate contact between them. Cap 103 can be molded as a separate complete item. If desired, apertures 108 and 109 can be provided with transparent inserts which may insure greater security against ingress of extraneous moisture from outside the housing. By providing a tight fit between the end 105 of housing 100 and the protruding sample collector 106, the application of sample to the protruding member will not result in sample entering the device directly and by-passing collector 106. Collector 106 therefore provides the sole route of access for the sample to the strip within the housing, and can deliver sample to the strip in a controlled manner. The device as a whole therefore combines the functions of sampler and analyser.

The porous sample receiving member can be made from any bibulous, porous or fibrous material capable of absorbing liquid rapidly. The porosity of the material can be unidirectional (i.e., with pores or fibres running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used.

It can be advantageous to pre-treat the member with a surface-active agent during manufacture, as this can reduce any inherent hydrophobicity in the member and therefore enhance its ability to take up and deliver a moist sample rapidly and efficiently. Porous sample receiving members can also be made from paper or other cellulosic materials, such as nitro-cellulose. Materials that are now used in the nibs of so-called fiber tipped pens are particularly suitable and such materials can be shaped or extruded in a variety of lengths and cross-sections appropriate in the context of the invention.

The material comprising the porous receiving member should be chosen such that the porous member can be saturated with aqueous liquid within a matter of seconds. The liquid must thereafter permeate freely from the porous sample receiving member into the macroporous body.

If present, the “control” zone can be designed merely to convey an unrelated signal to the user that the device has worked. For example, the control zone can be loaded with an antibody that will bind to the labelled reagent, e.g. an “anti-mouse” antibody if the labelled reagent is an antibody that has been derived using a murine hybridoma, to confirm that the sample has permeated the test strip. Alternatively, the control zone can contain an anhydrous reagent that, when moistened, produces a color change or color formation, e.g. anhydrous copper sulphate which will turn blue when moistened by an aqueous sample. As a further alternative, a control zone could contain immobilized analyte which will react with excess labelled reagent from the first zone. As the purpose of the control zone is to indicate to the user that the test has been completed, the control zone should be located downstream from the detection zone in which the desired test result is recorded. A positive control indicator therefore tells the user that the sample has permeated the required distance through the test device.

The label can be any entity the presence of which can be readily detected. Preferably the label is a direct label, ie. an entity which, in its natural state, is readily visible either to the naked eye, or with the aid of an optical filter and/or applied stimulation, e.g. UV light to promote fluorescence. For example, minute colored particles, such as dye sols, metallic sols (e.g. gold), and colored latex particles, are very suitable. Of these options, colored latex particles are most preferred. Concentration of the label into a small zone or volume should give rise to a readily detectable signal, e.g. a strongly-colored area. This can be evaluated by eye, or by instruments if desired.

Indirect labels, such as enzymes, e.g., alkaline phosphatase and horse radish peroxidase, can be used but these usually require the addition of one or more developing reagents such as substrates before a visible signal can be detected. Such additional reagents can be incorporated in the porous solid phase material or in the macroporous body, or in the sample receiving member if present, such that they dissolve or disperse in the aqueous liquid sample. Alternatively, the developing reagents can be added to the sample before contact with the porous material or the porous material can be exposed to the developing reagents after the binding reaction has taken place.

Coupling of the label to the specific binding reagent can be by covalent bonding, if desired, or by hydrophobic bonding. Such techniques are commonplace in the art. In some embodiments, where the label is a direct label such as a colored latex particle, hydrophobic bonding is preferred.

It is essential that the labelled reagent migrates with the liquid sample as this progresses to the detection zone. The flow of sample continues beyond the detection zone and sufficient sample is applied to the porous carrier material in order that this may occur and that any excess labelled reagent which does not participate in any binding reaction in the detection zone is flushed away from the detection zone by this continuing flow. Optionally, an absorbant “sink” can be provided at the distal end of the carrier material. The absorbent sink may comprise, for example, Whatman 3 MM chromatography paper, and should provide sufficient absorptive capacity to allow any unbound conjugate to wash out of the detection zone. As an alternative to such a sink it can be sufficient to have a length of porous solid phase material which extends beyond the detection zone.

The porous carrier material is in the form of a strip or sheet to which during manufacture of the device, one or more reagents can be applied in special distinct zones. During use, the liquid sample is allowed to permeate through the sheet or strip from one side or end to another.

If desired, a device according to the invention can incorporate two or more discrete bodies of porous solid phase carrier material, e.g. separate strips or sheets, each carrying immobilised reagents. These discrete bodies can be arranged in parallel, for example, such that a single application of liquid sample to the device initiates sample flow in the discrete bodies simultaneously. The separate analytical results that can be determined in this way can be used as control results, or if different reagents are used on the different carriers, the simultaneous determination of a plurality of analytes in a single sample can be made. Alternatively, multiple samples can be applied individually to an array of carriers and analysed simultaneously.

The porous solid phase material is nitrocellulose sheet having a pore size of at least about 1 micron, or greater than about 5 microns, or about 8-12 microns. Very suitable nitrocellulose sheet having a nominal pore size of up to approximately 12 microns, is available commercially from Schleicher and Schuell GmbH.

Preferably, the nitrocellulose sheet is “backed”, e.g. with plastics sheet, to increase its handling strength. This can be manufactured easily by forming a thin layer of nitrocellulose on a sheet of backing material. The actual pore size of the nitrocellulose when backed in this manner will tend to be, lower than that of the corresponding unbacked material.

Alternatively, a pre-formed sheet of nitrocellulose can be tightly sandwiched between two supporting sheets of solid material, e.g. plastics sheets. The flow rate of an aqueous sample through the porous solid phase material should be such that in the untreated material, aqueous liquid migrates at a rate of 1 cm in not more than 2 minutes, but slower flow rates can be used if desired.

The spatial separation between the macroporous body and the detection zone, and the flow rate characteristics of the porous carrier material, can be selected to allow adequate reaction times during which the necessary specific binding can occur. Further control over these parameters can be achieved by the incorporation of viscosity modifiers (e.g. sugars and modified celluloses) in the sample to slow down the reagent migration.

Preferably, the immobilized binding reagent in the reagent zone is impregnated throughout the thickness of the carrier in the reagent zone (e.g. throughout the thickness of the sheet or strip if the carrier is in this form). Such impregnation can enhance the extent to which the immobilized reagent can capture any analyte or labelled reagent, present in the migrating sample.

Reagents can be applied to the porous carrier material in a variety of ways. Various “printing” techniques have previously been proposed for application of liquid reagents to carriers, e.g. micro-syringes, pens using metered pumps, direct printing and ink-jet printing, and any of these techniques can be used in the present context. To facilitate manufacture, the carrier (e.g. sheet) can be treated with the reagents and then subdivided into smaller portions (e.g. small narrow strips each embodying the required reagent-containing zones) to provide a plurality of identical carrier units.

### Example 6—Lateral Flow Assay for Unfolded p53 in a Blood Sample

A sheet (1.4 mm thick) of commercially-available, detergent pre-treated, macroporous polyethylene having a pore size of about 100 microns is saturated with an aqueous suspension of colored latex particles (prepared as described in GB 2204398A) of particle size about 0.4 microns. The latex particles carry U-p53 binding agent (such as the 2D3A8 antibody). The solution contains 3% BSA and 4% sugar. The sheet is freeze-dried and cut into portions each 6×12 mm, having a liquid capacity of about 50 μL. The sheets are used as strips for the reagent zone in a lateral flow device as described above. The test strip has a sample loading zone which contains 10 μl of 90% pure acetonitrile, a reagent zone containing 15 μg amount of Up53 binding agent (the 2D3A8 antibody), and a detection zone containing 30 μg amount of secondary antibody such as anti-IgM (or anti-IgG2a, anti-IgG2b, anti-IgG3) that is immobilized on to the detection strip. In some embodiments, the secondary antibody is labeled with biotin which is then immobilized on the detection strip at the capture zone or detection zone by means of streptavidin embedded in the strip.

### Example 7—Detection of Onset of Dementia Using the Device

A blood sample is obtained from a subject who has no symptoms of dementia and may or may not have a family history of dementia. The blood sample is obtained by pricking the index finger with a pin and squeezing the finger until a single drop of blood appears (e.g., 50-100 ul). The blood sample from the subject is applied onto the sample zone of the device. If the blood sample contains unfolded p53 protein (U-p53^{AZ}), then a complex with the latex bead-labeled unfolded/misfolded-p53 binding agent (the 2D3A8 antibody) will form and the complex will migrate along the flow path to the detection zone, where it will be immobilized by the capture agent such as secondary antibody (anti-IgM) which binds to the U-p53-antibody (Up53 binding agent) and a colored line indicating the presence of unfolded U-p-53 protein in the blood sample. The limits of detection of the analyte U-p53AZ in a liquid sample is a concentration of U-p53AZ at or higher than 600±100 pg/ml.

1-4 minutes after the sample is applied to the lateral flow device, a color line in the detection zone may form. If no colored line is visible in the detection zone, then this represents a negative result in which unfolded-p53 (preferentially U-p53^{AZ}) p53 is not visually detected. If a colored line forms in 1-4 minutes, this represents a positive result and indicates that the subject is at risk for onset of dementia.

Where a positive result is visible, the device thus provides a risk assessment that the subject has a likelihood of developing symptoms of dementia within the next 6-10 years. The subject can then follow up with the physician to undergo further testing such as PET or MRI scans.

The subject can optionally undergo dementia management by undertaking one or more actions. These actions include but not limited to (a) Mentally stimulating activities, such as reading, solving puzzles and playing word games, (b) Being physically and socially active, (c) Avoid smoking, (d) Treat high blood pressure, high cholesterol, diabetes, and lower high body mass index (BMI), (e) Increase quality of sleep and (f) Maintain healthy diet and reduce vitamin D deficiency.

The subject can then repeat the test periodically, for example, after 6 months, after one year and longer periods of time, to see if the device produces a negative result that would indicate that the levels of U-p53 protein have decreased below the threshold of 500 pg/ml.

The same process can be replicated for a subject who has no symptoms of dementia but has a family history of dementia. The same process can be replicated for a subject who has no symptoms of dementia but have a genetic predisposition to developing dementia due to abnormal expression patterns, or mutations in, or have variants, of one or more genes selected from the group consisting of APOE4, ABCA7, CLU, CR1, PICALM, PLD3, TREM2, SORL1, APP, PSEN1 and PSEN2.

### Example 8—Diagnosis of the Presence of Dementia Using the Device

As described in Example 5A sheet (1.4 mm thick) of commercially-available, detergent pre-treated, macroporous polyethylene having a pore size of about 100 microns is saturated with an aqueous suspension of colored latex particles (prepared as described in GB 2204398A) of particle size about 0.4 microns. The latex particles carried Unfolded p53 binding agent. The solution also contained 3% BSA and 4% sugar. The sheet is freeze-dried and cut into portions each 6×12 mm, having a liquid capacity of about 50 μL. The sheets are used as test strips in any one of the devices as described above.

A blood sample is obtained from a subject who exhibits one or more symptoms associated with dementia. The blood sample is applied onto the sample zone of the device as described in Example 5. Where no colored line (signal) forms in the detection zone, this indicates that the subject does not have dementia. Where the subject has symptoms that are associated with dementia and yet has a negative result (no observable signal), this indicates that the subject's symptoms are due to a neurological disease other than dementia, for example, Parkinson's disease or multiple sclerosis. The formation of a colored line (a signal) indicates a positive result and confirms that the subject's one or more symptoms are indeed due to dementia. These results thus indicate that the subject must follow up with the physician so that more extensive testing, such as PET or MRI scans, and can also undergo proactive treatment to reduce symptoms of dementia.

1. A device for detecting the presence of unfolded p53 protein in a biological sample, the device comprising:
a test strip defining a flow path comprising a sample zone, a reagent zone, and detection zone:
wherein said sample zone is positioned along said flow path, said sample zone comprising a loading port for introducing said biological sample in liquid form into the flow path;
wherein said reagent zone is positioned along the flow path downstream from said sample zone, said reagent zone comprising a first binding agent which is mobilizable by flow of said sample along said flow path, said first binding agent comprising a detectable label and being capable of specifically binding unfolded p53 protein to form a first complex; and
wherein said detection zone is positioned downstream from said reagent zone along said flow path, and wherein flow of said sample along said flow path mobilizes said first binding agent to said detection zone;
wherein said first binding agent comprises an antibody or fragment thereof comprising heavy chain CDRs 1-3 and light chain CDRs 1-3, wherein said heavy chain CDRs 1-3 comprise heavy chain CDR1 (SEQ ID NO: 8), CDR2(SEQ ID NO: 9), and CDR3(SEQ ID NO: 10) and wherein said light chain CDRs 1-3 comprise light chain CDR1(SEQ ID NO:11), CDR2 (SEQ ID NO: 12) and CDR3(SEQ ID NO: 13), and
wherein formation of said first complex generates a detectable signal in said detection zone, indicating the presence of unfolded p53 protein in said biological sample at a concentration at or above 600±100 pg/ml.

2. A device for detecting the presence of unfolded p53 protein in a biological sample, the device comprising:
a test strip defining a flow path comprising a sample zone, a reagent zone, and capture zone;
wherein said sample zone is positioned along said flow path, said sample zone comprising a loading port for introducing said sample in liquid form into said flow path;
wherein said reagent zone is positioned downstream from said sample zone, said reagent zone comprising a first binding agent which is mobilizable by flow of said sample along said flow path, the first binding agent being capable of specifically binding unfolded p53 protein to form a first complex;
wherein said capture zone is positioned downstream of said reagent zone along said flow path, said capture zone comprising an immobilized second binding agent comprising a detectable label capable of binding to and thereby immobilizing said first complex;
wherein said first binding agent comprises an antibody or fragment thereof comprising heavy chain CDRs 1-3 and light chain CDRs 1-3, wherein said heavy chain CDRs 1-3 comprise heavy chain CDR1 (SEQ ID NO: 8), CDR2(SEQ ID NO: 9), CDR3(SEQ ID NO: 10) and wherein said light chain CDRs 1-3 comprise light chain CDR1(SEQ ID NO:11), CDR2(SEQ ID NO: 12) and CDR3(SEQ ID NO: 13), and
wherein immobilization of said first complex at said capture zone produces a detectable signal in said capture zone, said detectable signal indicating the presence of unfolded p53 protein in said biological sample at a concentration above 600±100 pg/ml.

3. The device of anyone of claims 1-2, further comprises a control zone comprising a secondary antibody that binds to the first binding agent to produce a detectable signal.

4. The device of anyone of the claims above, wherein said first binding agent comprises a heavy chain variable region and a light chain variable region, wherein said heavy chain comprises SEQ ID NO: 6 and said light chain comprises SEQ ID NO:7.

5. The device of anyone of the claims above, wherein said first binding agent comprises a heavy chain and a light chain, said heavy chain comprising SEQ ID NO: 4 and said light chain comprising SEQ ID NO: 5.

6. The device of anyone of the claims above, wherein said detectable label comprises one or more of colloidal gold particle, colloidal silver particle, colloidal platinum particle, latex beads, latex nanocomposites, magnetic beads. Quantum dots, fluorescent dyes, ruthenium complexes, nanocomposite particles and paramagnetic labels.

7. The device of anyone of the claims above, wherein said device further comprises an accelerant positioned alongside the longitudinal plane of the test strip that helps disperse the liquid sample across the flow path.

8. The device of claim 7, wherein said accelerant comprises a heating strip positioned alongside the longitudinal plane of the test strip effective to apply sufficient heat to the test strip so as to uniformly disperse liquid sample along the flow path.

9. The device of claim 7, wherein said accelerant comprises a magnetic strip positioned alongside the longitudinal plane of the test strip that applies magnetic field to ensure uniform dispersal of liquid sample across the flow path.

10. The device of claim 9, wherein said magnetic field is a constant magnetic field.

11. The device of claim 9, wherein said magnetic field is a dynamic magnetic field or a constant magnetic field.

12. A method for detecting the onset of dementia in a subject who does not exhibit one or more symptoms of dementia, the method comprising:
contacting a biological sample from said subject with the device of claim 1 or 2 by introducing said sample in liquid form into said loading port of said sample zone of said device; and
observing said detection zone of said device for a signal;
wherein visualization of a signal in said detection zone indicates the onset of dementia in said subject.

13. A method for confirming the presence of cognitive decline due to dementia in a subject who exhibits one or more symptoms of dementia, the method comprising:
contacting a biological sample from said subject with the device of claim 1 or 2 by introducing said sample in liquid form into said loading port of said sample zone of said device; and
observing said detection zone of said device for a signal;
wherein visualization of a signal in said detection zone confirms the presence of dementia in said subject.

14. The method of anyone of claims 12-13, wherein said observation of said signal in said detection zone indicates the presence of unfolded p53 protein in said biological sample at a concentration at or above 600±100 pg/ml.

15. The method of anyone of claims 12-14, wherein said symptoms comprise one or more of a cognitive and/or a psychological characteristic of dementia.

16. The method of claim 15, wherein a said cognitive change comprises memory loss, diminution in one's ability to communicate, diminution in one's visual and spatial abilities, diminution in one's ability to reason and problem solve, diminution in one's ability to handle complex tasks, to plan, to organize, to coordinate complex tasks, to perform motor functions when compared with an individual who has normal levels of unfolded p53 protein.

17. The method of claim 15, wherein said psychological change comprises one or more of depression, anxiety, paranoia, agitation, and hallucinations.

18. The method of anyone of claims 12-14, wherein said subject has a family history of dementia.

19. The method of anyone of claims 12-14, wherein said subject is genetically predisposed to have dementia.

20. The method of claim 19, wherein said subject has abnormal expression of or has a genetic alteration that affects the function of one or more genes selected from the group consisting of APOE4, ABCA7, CLU, CR1, PICALM, PLD3, TREM2, SORL1, APP, PSEN1 and PSEN2.

21. The method of anyone of the claims 12-20, wherein said sample is a blood sample.

22. The device of anyone of claims 1-11, wherein said sample is a blood sample.

23. The device of claim 3, wherein said secondary antibody is anti-IgM antibody.

24. A device comprising a macroporous body containing in the dry state a labelled specific binding reagent comprising an antibody or fragment thereof comprising heavy chain CDRs 1-3 and light chain CDRs 1-3, wherein said heavy chain CDRs 1-3 comprise heavy chain CDR1 (SEQ ID NO: 8), CDR2(SEQ ID NO: 9), and CDR3(SEQ ID NO: 10) and wherein said light chain CDRs 1-3 comprise light chain CDR1(SEQ ID NO:11), CDR2 (SEQ ID NO:12) and CDR3(SEQ ID NO: 13), wherein said labelled specific binding reagent is freely soluble or dispersible upon contact with an aqueous sample.

25. A method of detecting unfolded p53 (U-p53^{AZ}) in a biological sample of a subject, comprising the steps of:
contacting said biological sample of said subject with the device of claim 1 or 2 by introducing the sample in liquid form to the loading port of the sample zone and observing the detection zone for the presence of absence of a signal,
wherein the presence of a signal in the detection zone indicates the presence of U-p53^{AZ} in said sample, and the absence of a signal in the detection zone indicates the absence of U-p53^{AZ} in the sample.

26. The method claim 25, wherein the presence of U-p53^{AZ} in said sample when said sample is from a subject who displays no indicators of dementia signifies an risk of developing progressive dementia leading to AD.

27. The method claim 25, wherein the presence of U-p53AZ in said sample when said sample is from a subject who displays five or more indicators of dementia signifies progressive dementia leading to AD.

28. The method of claim 26 or 27, wherein said five indicators of dementia are selected from the group comprising: memory loss, hallucination, paranoia, aggressive behavior, depression, difficulty communicating, difficulty with visual and spatial abilities, difficulty with reasoning and problem-solving, difficulty handling complex tasks, difficulty with planning and organizing, difficulty with coordination and motor functions, agitation, confusion and disorientation.
